(19) United States
(12) Patent Application Publication
(10) Pub. No.: US 2021/0401826 A1
(43) Pub. Date: Dec. 30, 2021

(54) ABUSE DETERRENT PHARMACEUTICAL FORMULATIONS

(71) Applicant: AVEKSHAN, LLC., Alachua, FL (US)

(72) Inventors: Danny O. HELTON, Alachua, FL (US); John CRAN, Alachua, FL (US); Chris Daniel WILEY, Alachua, FL (US)

(21) Appl. No.: 17/267,323

(22) PCT Filed: Aug. 13, 2019
(86) PCT No.: PCT/US2019/046366

§ 371 Date: Feb. 9, 2021

Related U.S. Application Data
(60) Provisional application No. 62/718,165, filed on Aug. 13, 2018.

(51) Int. Cl. A61K 31/485; A61K 31/4458; A61K 31/137; A61K 31/165; A61K 31/5375; A61K 9/16

(52) U.S. Cl. CPC A61K 31/485; A61K 31/4458; A61K 31/137; A61K 9/167; A61K 31/5375; A61K 9/1635; A61K 31/165

(57) ABSTRACT

In various aspects and embodiments, the present invention provides abuse deterrent pharmaceutical formulations, and methods of making the same. The abuse deterrent formulations can comprise a CNS stimulant (particularly one that is addictive or prone to be abused) and an opioid receptor antagonist. These agents are bound by one or more pharmaceutically acceptable resins (e.g., ion exchange resins) to limit their potential to be separated by a potential abuser.

### CROSS-REFERENCE TO RELATED APPLICATIONS

This application is a national stage application of PCT/US2019/046366 filed Aug. 13, 2019, which claims the benefit of and priority to U.S. Provisional Patent Application No. 62/718,165 filed Aug. 13, 2018, the content of which is hereby incorporated by reference in its entirety.

### TECHNICAL FIELD

The present disclosure in various aspects relates to abuse deterrent pharmaceutical formulations of active agents that are otherwise susceptible to abuse.

### BACKGROUND

While stimulant medicines are documented as effective treatments of, for example, ADHD, persistent concerns remain about their abuse potential that greatly impact their use in clinical practice. Combining stimulant medicines, such as methylphenidate, with an opioid blocker such as naltrexone, can reduce the feelings of euphoria some patients experience when taking these medicines. However, if the stimulant can be isolated from the opioid blocker, the mitigating effect of the opioid antagonist can be eliminated by a drug abuser.

Accordingly, there remains a need for abuse deterrent pharmaceutical formulations of these active agents.

### SUMMARY

In various aspects and embodiments, the present invention provides abuse deterrent pharmaceutical formulations, and methods of making the same. The abuse deterrent formulations can comprise a CNS stimulant (particularly one that is addictive or prone to be abused) and an opioid receptor antagonist. These agents are bound by one or more pharmaceutically acceptable resins (e.g., ion exchange resins) to limit their potential to be separated by a potential abuser.

For example, the one or more resins are selected to bind the CNS stimulant and the opioid receptor antagonist in the presence of at least one solvent, where the CNS stimulant and the opioid receptor antagonist have substantially different solubilities in the at least one solvent. These differences in solubility would otherwise provide a potential abuser with an opportunity to separate/isolate the stimulant from the opioid antagonist.

In some embodiments, the resin prevents the isolation or “washing out” of the addictive active agent from the formulation when the formulation is contacted with a solvent in which the addictive active agent is soluble but the opioid receptor antagonist is much less soluble. Alternatively, in some embodiments, the resin prevents the isolation or “washing out” of the opioid receptor antagonist from the formulation when the formulation is contacted with a solvent in which the opioid receptor antagonist is soluble but the addictive active agent is much less soluble. The formulation further allows for both the CNS stimulant and the opioid receptor antagonist to be released in the presence of gastric fluids (e.g., gastric acid, gastric juice, or stomach acids) or simulated gastric fluid (SGF), and the formulation can be designed to have various release profiles, such as an immediate release profile or an intermediate or extended release profile.

In an exemplary embodiment, the CNS stimulant is methylphenidate, or a pharmaceutically acceptable salt thereof, and the opioid receptor antagonist is naltrexone, or a pharmaceutically acceptable salt thereof.

The resin(s) is/are selected to bind both the CNS stimulant and the opioid receptor antagonist in one or more solvents where the two agents have substantially different solubility. For example, one agent is soluble in the solvent and the other is insoluble or weakly soluble in the solvent. The resin(s) may bind the two agents in the presence of at least 2, 3, 4, or 5 different solvents where the agents have substantially different solubility. The agents can be bound to the same resin, or different resins.

The resin(s) can be selected from various commercially available resins, including pharmaceutically acceptable ion exchange resins. In various embodiments, the resin is a weak acid cation ion exchange resin, a strong acid cation ion exchange resin, a weak basic anion ion exchange resin, a strong basic anion ion exchange resin, or a medium basic anion exchange resin. The resin is generally insoluble in water and in common solvents.

The solvent in which the two agents have substantially different solubility, and in which the resin(s) bind the active agents, can be selected from common solvents used in chemistry and/or common household solvents (including polar and non-polar solvents, protic and aprotic solvents, and organic solvents including oils).

In some embodiments, the CNS stimulant and the one or more opioid receptor antagonists are released from the resin in the presence of gastric fluids, which can be demonstrated using simulated gastric fluid. In various embodiments, the agents are released in gastric fluids or simulated gastric fluid with an immediate release profile. In some embodiments, the agents demonstrate an intermediate or extended release profile.

For an exemplary formulation, the stimulant is methylphenidate, or a pharmaceutically acceptable salt thereof, and the opioid receptor antagonist is naltrexone, or a pharmaceutically acceptable salt thereof. In some embodiments, an exemplary resin is a weakly acidic cation exchange resin, such as WK40L (DIAION), which has a polyacrylate backbone, and carboxylic acid groups as ionizable groups. For most solvents, including water, naltrexone (NTX) and methylphenidate (MPH) (or pharmaceutically acceptable salts thereof) have similar solubility (e.g., in the absence of the resin). However, for acetone, acetonitrile and isopropyl alcohol, NTX and MPH have differential solubility, whereby MPH is soluble while NTX is insoluble (or sparingly soluble) (e.g., in the absence of the resin). As such, this creates the opportunity for a potential drug abuser to “wash out” or isolate MPH from a mixture of the two agents. However, when bound to a weakly acidic cation exchange resin, such as WK40L resin, both agents remain bound to the resin in the presence of acetone, acetonitrile, and isopropyl alcohol (including for a period of 24 hours), while allowing for an immediate release profile in gastric fluids or simulated gastric fluid (SGF).

In various aspects and embodiments, the invention provides a method for making an abuse deterrent pharmaceutical formulation. In some embodiments, the method comprises loading a CNS stimulant and an opioid receptor antagonist onto one or more pharmaceutically acceptable resins. The one or more resins are identified as binding the CNS stimulant and the opioid receptor antagonist in the presence of at least one solvent where the CNS stimulant and the opioid receptor antagonist have substantially different solubilities in the at least one solvent. Thus, the solubility of the CNS stimulant and the opioid receptor antagonist are evaluated across a panel of solvents. Solvents are selected where the solubility of the two agents are substantially different, and one or more of these solvents are used to select the desired resin. A panel of resins can be tested for the ability to bind the two agents in the presence of the identified solvents. The panel of resins and solvents can include those described herein, as well as others. In some embodiments, the agents remain bound to the selected resin(s) in the presence of identified solvent(s) for at least about 8 hours, or for at least about 12 hours, or for at least about 24 hours, and at pH and temperatures where the CNS stimulant is stable.

The formulation of the present disclosure can take a variety of forms suitable for oral administration, including capsules and tablets, and may include various other inactive ingredients, including excipients and binders known in the art.

Other aspects and embodiments will be apparent from the following detailed description.

### BRIEF DESCRIPTION OF THE FIGURES

FIG. 1A is a table showing the amount of naltrexone released (mg) from ion-exchange resins (WK10, WK11, WK40, WK100, WTO1S, CG50, and IRP-64) in a dissolution apparatus in simulated gastric fluid (SGF).

FIG. 1B is a table showing the percent of naltrexone released from ion-exchange resins (WK10, WK11, WK40, WK100, WTO1S, CG50, and IRP-64) in a dissolution apparatus in simulated gastric fluid (SGF).

FIG. 1C is a graph showing the percent of naltrexone released from ion-exchange resins (WK10, WK11, WK40, WK100, WTO1S, CG50, and IRP-64) over time (15, 30, 45, and 60 minutes) in a dissolution apparatus in simulated gastric fluid (SGF).

FIG. 2A is a table showing the amount of methylphenidate released (mg) from ion-exchange resins (WK10, WK11, WK40, WK100, WTO1S, CG50, and IRP-64) in a dissolution apparatus in simulated gastric fluid (SGF).

FIG. 2B is a table showing the percent of methylphenidate released from ion-exchange resins (WK10, WK11, WK40, WK100, WTO1S, CG50, and IRP-64) in a dissolution apparatus in simulated gastric fluid (SGF).

FIG. 2C is a graph showing the percent of methylphenidate released from ion-exchange resins (WK10, WK11, WK40, WK100, WTO1S, CG50, and IRP-64) over time (15, 30, 45, and 60 minutes) in a dissolution apparatus in simulated gastric fluid (SGF).

FIG. 3A is a table showing the release of methylphenidate and naltrexone from resins (WK11, WK40L, and CG-50 (type 1)) using water.

FIG. 3B is a table showing the release of methylphenidate and naltrexone from resins (WK11, WK40L, and CG-50 (type 1)) using acetone.

FIG. 3C is a table showing the release of methylphenidate and naltrexone from resins (WK11, WK40L, and CG-50 (type 1)) using acetonitrile.

FIGS. 4A and 4B show the release of Methylphenidate using resins WK10S and CM400/SS in Simulated Gastric Fluid (SGF) in milligrams (FIG. 4A) and percentage (FIG. 4B). FIGS. 4C and 4D show the release of Naltrexone using resins WK10S and CM400/SS in SGF in milligrams (FIG. 4C) and percentage (FIG. 4D).

### DETAILED DESCRIPTION

In various aspects and embodiments, the present invention provides abuse deterrent pharmaceutical formulations, and methods of making the same. The abuse deterrent formulations can comprise a CNS stimulant (particularly one that is addictive or prone to be abused) and an opioid receptor antagonist. These agents are bound by one or more pharmaceutically acceptable resins (e.g., ion exchange resins) to limit their potential to be separated by a potential abuser. For example, the one or more resins are selected to bind the CNS stimulant and the opioid receptor antagonist in the presence of at least one solvent, including common household solvents, where the CNS stimulant and the opioid receptor antagonist have substantially different solubilities in the solvent(s). These differences in solubility would otherwise provide a potential abuser with an opportunity to separate/isolate the stimulant from the opioid antagonist. The resin thereby prevents the isolation or “washing out” of the addictive active agent from the formulation when the formulation is contacted with a solvent in which the addictive active agent is soluble but the opioid receptor antagonist is much less soluble.

In various embodiments, the formulation allows for the CNS stimulant and the opioid receptor antagonist to be released in the presence of gastric fluids (e.g., gastric acid, gastric juice, or stomach acids), which can be demonstrated using simulated gastric fluid (SGF), and the formulation can be designed to have various release profiles, such as an immediate release profile or an intermediate or extended release profile.

Exemplary CNS stimulants that can be formulated in accordance with embodiments of the invention include methylphenidate (MPH), amphetamine, benzphetamine, dextroamphetamine, dexmethylphenidate, diethylpropion, lisdexamfetamine, methamphetamine, armodafinil, modafinil, phendimetrazine, and phentermine, or pharmaceutically acceptable salts thereof.

Exemplary opioid receptor antagonists to reduce feelings of euphoria include naltrexone (NTX), nor-binaltorphimine (norBNI), nalmefene, nalodeine, samidorphan, naloxone, and 6β-Naltrexol, or pharmaceutically acceptable salts thereof.

In an exemplary embodiment, the CNS stimulant is methylphenidate, or a pharmaceutically acceptable salt thereof, and the opioid receptor antagonist is naltrexone, or a pharmaceutically acceptable salt thereof. In some embodiments, the dose of the methylphenidate or salt thereof in the pharmaceutical composition is from 10 to 120 mg, such as from 20 to 100 mg, or about 60 mg in some embodiments. In some embodiments, the dose of naltrexone or salt thereof is from about 1 mg to about 100 mg, such as from about 10 mg to about 80 mg, or from 25 mg to about 75 mg.

The resin(s) is/are selected to bind both the CNS stimulant and the opioid receptor antagonist in one or more solvents where the two agents have substantially different solubility. For example, one agent is soluble in the solvent and the other is insoluble or weakly soluble in the solvent. As used herein, the term “substantially different solubility” means that one of the agents demonstrates at least three times the solubility in the particular solvent than the other agent. In various embodiments, a resin(s) is/are selected which bind the two agents in one or more solvents in which one agent has at least about 5 times higher solubility than the other agent, or at least 10 times higher solubility than the other agent. In various embodiments, the resin(s) bind both agents in solvents where one agent has a solubility of at least about 0.5 mg/mL, or at least about 1 mg/mL, or at least about 1.5 mg/mL, or at least about 2 mg/mL, or at least about 5 mg/ml, or at least about 10 mg/mL; and the other has a solubility of less than about 0.4 mg/mL, or less than about 0.2 mg/mL, or less than about 0.1 mg/mL. The resin(s) may bind the two agents in the presence of (independently) at least 2, 3, 4, or 5 different solvents where the agents have substantially different solubility. The agents can be bound to the same resin, or different resins.

In some embodiments, a substantially different solubility is present when the CNS stimulant is soluble in the solvent and the opioid receptor antagonist is insoluble or weakly soluble in the same solvent. In some embodiments, a substantially different solubility is present when the opioid receptor antagonist is soluble (e.g., greater than about 1.0 mg/mL) in the solvent and the CNS stimulant is insoluble or weakly soluble (e.g., less than about 0.4 mg/mL) in the same solvent, or less than about 0.2 mg/ml or less than about 0.1 mg/mL.

In some embodiments, the solubility of the CNS stimulant is greater than about 1.0 mg/mL in a solvent and the solubility of the opioid receptor antagonist is less than about 0.1 mg/mL in the same solvent. In some embodiments, the CNS stimulant has a solubility of between about 1.0 and 0.5 mg/mL in a solvent and the solubility of the opioid receptor antagonist is less than about 0.1 mg/mL in the same solvent. In some embodiments, the CNS stimulant has a solubility of greater than about 1.0 mg/mL in the solvent and the solubility of the opioid receptor antagonist is between about 0.1 and 0.5 mg/mL in the same solvent. The resin(s) can be selected from various commercially available resins, including pharmaceutically acceptable ion exchange resins. In various embodiments, the resin is a weak acid cation ion exchange resin, a strong acid cation ion exchange resin, a weak basic anion ion exchange resin, a strong basic anion ion exchange resin, or a medium basic anion exchange resin. The resin is generally insoluble in water and in common solvents.

The resin will generally comprise a polymer backbone with functional groups for binding active agents. Exemplary resins can comprise a polymer or copolymer of one or more of acrylate, methacrylate, ethylene, divinylbenzene (DVB), and polystyrene. For example, the resin may comprise a polymer backbone selected from acrylic, methacrylic, methacrylic-DVB copolymer, styrene-DVB co-polymer; an acryl-DVB copolymer, an acrylic ester, and others. In various embodiments, the polymer is crosslinked (e.g., at from about 1 to 5% crosslinking density). The resin comprises one or more functional groups for binding active agent, which can be selected from one or more of primary amine, secondary amine, tertiary amine, quaternary amine (e.g., trimethylammonium), carboxylic acid, sulfonic acid, thiol, imiodiacetic acid, aminophosphonic acid, thiourea, bis-picolylamine, dithiocarbamate, thiouronium, amidoxime, and N-methyl glucamine. In some embodiments, the functional groups are in various protonation states, thereby modulating the charge of the function group.

In various embodiments, the resin has a size in the range of about 50 and 500 mesh. For example, in various embodiments, the resin has a particle size distribution within the range of about 10 μM to about 1000 μm, or from about 50 to 750 μm, or from about 100 to 500 μm. In some embodiments, the resin is between about 50-100 mesh, about 100-150 mesh, about 100-200 mesh, about 150-250 mesh, about 200-300 mesh, about 200-400 mesh, about 250-350 mesh, about 300-400 mesh, or about 400-500 mesh. In some embodiments, the resin is in the form of a powder, a gel, or beads. In some embodiments, resin beads are monodispersed (i.e., having beads of uniform size) or heterodispersed (i.e., having beads of various sizes). The resin is generally porous, and may be macroporous or microporous. In some embodiments, the resin is isoporous, as these terms are understood in the art.

In some embodiments, the resin is a weak acid cation ion exchange resin, and is based on a crosslinked polyacrylic polymer with a carboxylic acid functional group.

In some embodiments, the resin is a weak acid cation ion exchange resin, and is based on a crosslinked polyacrylic polymer with a carboxylate functional group.

In some embodiments, the resin is a strong acid cation ion exchange resin, and is based on a crosslinked polystyrene with a sulfonic acid functional group.

In some embodiments, the resin is a weak acid cation ion exchange resin, and is based on crosslinked methacrylic with a carboxylic acid functional group.

In some embodiments, the resin is a weak acid cation ion exchange resin, and is based on crosslinked methacrylic with a carboxylate functional group.

In some embodiments, the resin is a strong acid cation ion exchange, and is based on a crosslinked styrene-DVB co-polymer with a sulfonate functional group.

Exemplary resins may be selected from DIAION's WK10, WK10S, WK11, WK100, WTO1S (which are all methacrylic type resins), DIAION's WK40L (acrylic type), AMBERLITE CG50 (type 1), AMBERLITE IRP-69 or IRP-64, DOWEX 50WX2-100, DOWEX 50WX2-400, DOWEX 50WX4-100, and DOWEX 50WX4-400.

In various embodiments, the present abuse deterrent pharmaceutical formulation further comprises at least one pharmaceutically acceptable gelling agent (or viscosity enhancer). As used herein a “gelling agent” or “viscosity enhancer” refers to an agent that imparts a gel-like or thickening quality to a tampered dosage form. In some embodiments, the addition of moisture or liquid to the gelling agent in the dosage form imparts a viscosity unsuitable for administration by parenteral and/or nasal administration to a solubilized mixture formed when the dosage form is crushed and/or mixed with an aqueous liquid. In some embodiments, the active pharmaceutical ingredient that is suspended in high viscosity solutions is unsuitable for abuse via intravenous injections.

In some embodiments the gelling agent is a gum, wherein the gum is selected from acacia gum, agar gum, tragacanth gum, guar gum, xanthan gum, locust bean gum, tara gum, karaya gum, gellan gum, welan gum, and rhamsan gum. In some embodiments, the gelling agent is colloidal anhydrous silica/xanthan gum.

In some embodiments, the gelling agent is selected from curdlan, furcelleran, agar, glucomannans, egg white powder, lacto albumin, soy protein, Jargel, sugars or sugar derived alcohols, such as mannitol, sorbitol, starch and starch derivatives, cellulose derivatives, such as microcrystalline cellulose, sodium cahoxymethyl cellulose, methylcellulose, ethyl cellulose, hydroxyethyl cellulose, hydroxypropyl cellulose, and hydroxypropyl methylcellulose, attapulgites, bentonites, dextrins, alginates, carrageenan, pectin, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and combinations thereof.

The solvent in which the two agents have substantially different solubility, and in which the resin(s) bind the active agents, can be selected from any common solvents used in chemistry and/or common household solvents (including polar and non-polar solvents, protic and aprotic solvents, and organic solvents including oils). Exemplary solvents include acetone, acetonitrile, alcohols (e.g., methanol, ethanol, and isopropyl alcohol), methyl ethyl ketone, alkanes (e.g., hexane), dimethylformamide (DMF), dimethyl sulfoxide (DMSO), HCl, aqueous NaOH, toluene, acetic acid, and ethyl acetate. Other solvents include those that might be easily available to a potential abuser, including but not limited to, water, vinegar, soft drinks (e.g., carbonic acid), baking soda, vegetable oil, xylene, mineral spirits, turpentine, lye, various potential hydrocarbon solvents (e.g., naphtha), and other publically available solvents (e.g., found in hardware stores, pool suppliers, etc).

In some embodiments, the CNS stimulant and the one or more opioid receptor antagonists are released from the resin in the presence of gastric fluids (e.g., gastric acid, gastric juice, or stomach acids), which can be demonstrated using SGF. In various embodiments, the agents are released in simulated gastric fluid with an immediate release profile. For example, an immediate release profile provides for at least about 70% of the agents to be released within about 1 hour. In some embodiments, at least 70% of the agents are released in the presence of SGF within about 45 minutes, or within about 30 minutes, or within about 15 minutes.

In some embodiments, the agents demonstrate an intermediate or extended release profile, meaning that greater than 30% of the active agent, or greater than 50% of the active agent, or greater than 70% of the active agent have not been released after 1 hour in SGF. In these embodiments, at least 70% of the active agents are released by about 90 minutes in SGF. In some embodiments, at least about 70% of the active agent is released after about 90 minutes in simulated gastric fluid (SGF), or about 120 minutes, or about 180 minutes, in SGF.

For an exemplary formulation, the stimulant is methylphenidate or amphetamine, or pharmaceutically acceptable salts thereof, and the opioid receptor antagonist is naltrexone, or a pharmaceutically acceptable salt thereof. In such embodiments, an exemplary resin is a weakly acidic cation exchange resin, such as WK40L (DIAION), which has a polyacrylate backbone and carboxylic acid groups as ionizable groups. In some embodiments, the resin is a methacrylic-type resin which is a weak acid cation exchange resin having carboxylic acid functionalities. Methacrylic-type resins that may be used include, for example, DIAION's WK10, WK10S, WK11, WK100, and WTO1S. In one embodiment, the resin is DIAION WK10S. In another embodiment, the resin is a copolymer of methacrylic acid and divinylbenzene, e.g., AMBERLITE IRP-64. For most solvents, including water, naltrexone (NTX) and methylphenidate (MPH) have similar solubility (e.g., in the absence of the resin). However, for acetone, acetonitrile and isopropyl alcohol, NTX and MPH have differential solubility, whereby MPH is soluble while NTX is insoluble (or sparingly) soluble (e.g., in the absence of the resin). As such, this creates the opportunity for a potential drug abuser to “wash out” or isolate MPH from a mixture of the two agents. However, when bound to a weakly acidic cation exchange resin, such as WK40L resin, both agents remain bound to the resin in the presence of acetone, acetonitrile, and isopropyl alcohol (including for a period of 24 hours), while allowing for an immediate release profile in gastric fluid or SGF.

In various aspects and embodiments, the invention provides a method for making an abuse deterrent pharmaceutical formulation. In some embodiments, the method comprises loading a CNS stimulant and an opioid receptor antagonist on to one or more pharmaceutically acceptable resins. The CNS stimulant and the opioid receptor antagonist have substantially different solubility in the presence of at least one solvent and the one or more resins are identified as binding the CNS stimulant and the opioid receptor antagonist in the presence of the at least one solvent. Thus, the solubility of the CNS stimulant and the opioid receptor antagonist are evaluated across a panel of solvents, to identify solvent(s) in which the solubilities of the two agents are substantially different. Solvents are selected where the solubility of the two agents are substantially different, and one or more of these solvents are used to select the desired resin. A panel of resins can be tested for the ability to bind the two agents in the presence of the identified solvents. The panel of resins and solvents can include those described herein, as well as others. In some embodiments, the selected resin binds each agent in the presence of identified solvent(s) for at least about 8 hours, or for at least about 12 hours, or for at least about 24 hours, and at pH and temperatures where the CNS stimulant is substantially stable.

The process for loading the active agents on to the resin can take a variable amount of time. In various embodiments, active agent is loaded onto the resin for from 1 hour to 24 hours, such as from about 2 hours to about 10 hours. In some embodiments, this loading step can be pH dependent. In some embodiments, a cation exchange resin is loaded with the active agents at a pH of about 4.5, about 5, about 5.5, about 6, or about 6.5. In some embodiments, an anion exchange resin is loaded with the active agents at a pH of about 7, about 7.5, about 8, or about 8.5. In some embodiments, an anion exchange resin is loaded with the active agents at a pH of about 7, about 6.5, about 6, about 5.5, about 5, about 4.5, about 4, about 3.5, about 3.

The formulation of the present disclosure can take a variety of forms suitable for oral administration, including capsules and tablets, and may include various other inactive ingredients, including excipients and binders known in the art.

In some aspects, the present disclosure relates to the treatment of a disorder or condition with the CNS stimulant. In some embodiments, the method of treatment comprises administering the formulation disclosed herein for a condition such as attention deficit hyperactivity disorder (ADHD), bipolar disorder, or depression. Because the agents are sufficiently released from the resin in gastric fluid or SGF, the resin does not impact the pharmaceutical effectiveness of the composition.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

### EXAMPLES

### Example 1. Solubility Study

This example shows that methylphenidate (MPH) can be isolated from naltrexone using a differential (selective) extraction using a solvent in which only one of the two drugs is significantly soluble.

The solubility of naltrexone and methylphenidate was evaluated in various commonly available solvents and household items, including water, cola soft drinks, ethanol, vinegar, nail polish remover, toluene, turpentine, and baking soda solution (see Table 1). The solubility results were determined by visual examination of each solution after warming and several hours of mixing. The solubility results are presented below in Table 1.

TABLE 1
Solubility Results (mg/ml)

| Solvent | Naltrexone HCl | Methylphenidate HCl |
|---|---|---|
| Water | >1.0 mg/mL | >1.0 mg/mL |
| Acetone | <0.1 mg/mL | >1.0 mg/mL |
| Isopropyl Alcohol | <0.1 mg/mL | >1.0 mg/mL |
| DMF | >1.0 mg/mL | >1.0 mg/mL |
| Ethanol | >1.0 mg/mL | >1.0 mg/mL |
| Methanol | >1.0 mg/mL | >1.0 mg/mL |
| Hexanes | <0.1 mg/mL | 0.1 mg/mL < x < |
| | | 0.5 mg/mL |
| Acetonitrile | 0.1 mg/mL < x < | >1.0 mg/mL |
| | 0.5 mg/mL | |
| Ethyl Acetate | <0.1 mg/mL | 0.1 mg/mL < x < |
| | | 0.5 mg/mL |
| DMSO | >1.0 mg/mL | >1.0 mg/mL |
| 0.1M Hydrochloric Acid | >1.0 mg/mL | >1.0 mg/mL |
| 0.1M Sodium Hydroxide | >1.0 mg/mL | >1.0 mg/mL |
| 40% Ethanol | >1.0 mg/mL | >1.0 mg/mL |
| Toluene | <0.1 mg/mL | <0.1 mg/mL |
| Vinegar | >1.0 mg/mL | >1.0 mg/mL |
| Coca-Cola | >1.0 mg/mL | >1.0 mg/mL |
| Canola Oil | <0.1 mg/mL | <0.1 mg/mL |
| Xylene | <0.1 mg/mL | <0.1 mg/mL |
| Methyl Ethyl Ketone | <0.1 mg/mL | 0.5 mg/mL < x < |
| | | 1.0 mg/mL |
| Mineral Spirits | <0.1 mg/mL | <0.1 mg/mL |
| Turpentine | <0.1 mg/mL | <0.1 mg/mL |
| Naphtha | <0.1 mg/mL | <0.1 mg/mL |
| 0.2% Baking Soda | >1.0 mg/mL | >1.0 mg/mL |
| 50:50 Acetone:H₂O | >1.0 mg/mL | >1.0 mg/mL |
| 25:75 Acetone:H₂O | >1.0 mg/mL | >1.0 mg/mL |

### Example 2. Isolation of Methylphenidate Using Acetone

As shown in Example 1 (see Table 1), acetone, which is commonly available in nail polish remover, was a selective solvent as acetone was a strong solvent for methylphenidate but a weak solvent for naltrexone (i.e., methylphenidate was significantly soluble in acetone (>1.0 mg/ml), while naltrexone was not (<0.1 mg/ml)).

In this example, equal amounts of methylphenidate and naltrexone (20 mg each) were combined and then separated. To separate the methylphenidate from the naltrexone, only materials representing commonly available household items were used, e.g., acetone (nail polish remover), water, small glass containers, a hot plate and a coffee filter. Three sources of acetone were used, all of which are available to the public, including lab grade acetone, nail polish remover, and acetone heavy-duty cleaner.

Conditions for the separation, such as acetone volume and extraction time, were tested and adjusted. Depending on the volume of acetone used, 73-92% of methylphenidate was extracted, dried at room temperature, then reconstituted in water, while up to 9500 of the naltrexone was removed from the 1:1 drug mixture. About 14 to 19 mg of the initial 20 mg of methylphenidate was isolated and reconstituted with 10000 water, with up to 9500 of the naltrexone removed. The results for separation of methylphenidate from naltrexone using acetone are presented in Table 2.

TABLE 2
Separation of Naltrexone (NTX) and Methylphenidate (MPH) Using Acetone

| | NTX | mg NTX | % NTX | MPH | mg MPH | % MPH |
|---|---|---|---|---|---|---|
| Source of Acetone | (mg/mL) | Recovered | Recovered | (mg/mL) | Recovered | Recovered |
| Nail Polish Remover | 0.14 | 2.76 | 13.8 | 0.91 | 18.3 | 91.4 |
| 50 mL (Prep. A) | | | | | | |
| Nail Polish Remover | 0.15 | 3.08 | 15.4 | 0.90 | 18.0 | 90.1 |
| 50 mL (Prep. B) | | | | | | |
| Nail Polish Remover | 0.06 | 1.12 | 5.6 | 0.73 | 14.6 | 73.0 |
| 20 mL (Prep. A) | | | | | | |
| Nail Polish Remover | 0.09 | 1.72 | 8.6 | 0.79 | 15.9 | 79.4 |
| 20 mL (Prep. B) | | | | | | |
| Acetone Cleaner | 0.13 | 2.68 | 13.4 | 0.92 | 18.4 | 92.0 |
| 50 mL (Prep. A) | | | | | | |
| Acetone Cleaner | 0.13 | 2.5 | 12.5 | 0.92 | 18.4 | 91.8 |
| 50 mL (Prep. B) | | | | | | |
| Acetone Cleaner | 0.05 | 1.02 | 5.1 | 0.79 | 15.9 | 79.5 |
| 20 mL (Prep. A) | | | | | | |
| Acetone Cleaner | 0.09 | 1.7 | 8.5 | 0.80 | 15.9 | 79.7 |
| 20 mL (Prep. B) | | | | | | |
| Lab Grade Acetone | 0.31 | 6.22 | 31.1 | 0.91 | 18.3 | 91.5 |
| 50 mL (Prep. A) | | | | | | |
| Lab Grade Acetone | 0.11 | 2.3 | 11.5 | 0.92 | 18.5 | 92.4 |
| 50 mL (Prep. B) | | | | | | |
| Lab Grade Acetone | 0.05 | 1.04 | 5.2 | 0.76 | 15.3 | 76.4 |
| 20 mL (Prep. A) | | | | | | |
| Lab Grade Acetone | 0.06 | 1.3 | 6.5 | 0.74 | 14.7 | 73.7 |
| 20 mL (Prep. B) | | | | | | |

The separation of NTX and MPH was accomplished with minimal effort. The assay using common household materials was a viable strategy to isolate methylphenidate from the combined NTX/MIPH mixture and consume it dissolved in water.

### Example 3. Methylphenidate Loading and Release Using Amberlite Resins

AMBERLITE resins were used as a possible formulation barrier to prevent the abuse strategy of differential (selective) extraction (described in Example 2). To evaluate the resin formulation strategy, six selected AMBERLITE resins were evaluated for their drug loading capacity and release profiles.

Resin loading for methylphenidate was examined. Six types of AMBERLITE resins were selected and conditioned based on literature guidance and manufacturer's recommendations. Methylphenidate drug loading was determined separately at pH 6.0 and pH 7.0 after about 4 hours of resin (˜200 mg) soaking in a solution of methylphenidate (1.0 mg/mL×20 mL) in an aqueous buffer. An earlier loading study demonstrated that pH 6-7 achieved greater than 90% drug loading, and longer loading time, up to 48 hours, did not result in significantly more loading.

Method for Loading Methylphenidate onto Resin

Resin Rinse and Wash: A Buchner Filtration apparatus was assembled and connected to vacuum source. For each resin type, 5.0±0.01 g of dry resin was weighed. The resins were transferred to filter media on Buchner/filtration apparatus and rinsed with five 50-mL (appx.) aliquots of DI water. The following resins were prepared: AMBERLITE CG50 (type 1) (50-150 μm particle size); AMBERLITE IRP-69 (50-150 μm particle size); DOWEX 50WX2-100 (2% DVB cross-linking, 50-100 mesh); DOWEX 50WX2-400 (2% DVB cross-linking, 200-400 mesh); DOWEX 50WX4-100 (4% DVB cross-linking, 50-100 mesh); and DOWEX 50WX4-400 (4% DVB cross-linking, 200-400 mesh).

Resin Activation: 0.1 M HCl and 0.1 M NaOH solutions were used. Using Buchner apparatus, each resin was activated by slowly rinsing with two 50-mL (appx.) aliquots of 0.1 M HCl solution, followed by two 50-mL (appx.) aliquots of NaOH. The rinse cycle was performed three times. 0.1 M NaOH wash used at the end of each cycle in order to activate resin per desired loading conditions. Filter media containing activated resin was transferred to oven to dry for 2 to 4 hrs at 35 to 45° C.

Resin Loading: A series of 50 mM sodium phosphate monobasic dihydrate pH buffer stock solutions were prepared. Each pH buffer solution series was used to evaluate AMBERLITE/DOWEX resin drug-loading conditions. NaOH was used to adjust pH of each solution to one of the following values: pH 6.0±0.1; pH 7.0±0.1; pH 8.0±0.1; or pH 9.0 0.1.

200 mg+2 mg of methylphenidate was added to 200 mL volumetric flask. Flask was brought to volume with the pH 6.0 sodium phosphate buffer and shake/stir until fully dissolved. This resulted in a 1.0 mg/mL stock reference solution. The previous steps were repeated for each pH sodium phosphate buffers used.

Test samples were prepared by labeling separate 20-ml vials with each resin type and pH buffer. 20-ml of buffer was transferred into each sample. The reference solutions were injected onto HPLC.

Test sample concentrations, in each respective solvent, were recorded before addition of AMBERLITE/DOWEX Resin using known concentrations of reference solutions. The concentration of each buffer series reference solution was recorded as the before (t=0 hr) concentration of all test samples in the same series. 200 mg+2 mg of dried, activated resins was transferred to test sample vials.

Resin and test samples were mixed by stirring at ambient temperature for at least 12 hr. Small, μl-quantity aliquots were sampled and quantitatively diluted at specified time points for HPLC analysis to gauge completeness of loading. Injected (5-20 μl TBD) of test samples, in each respective solvent/resin combination.

Calculated test sample concentration (C₂), in each respective solvent, after addition of AMBERLITE/DOWEX Resin and 5 hr loading time using peak areas of test sample (A₂) and reference solution (A₁), and the known concentration of reference solution (C₁): test sample concentration (mg/ml)=C₂=(C₁×A₂)/A₁.

For each respective solvent/resin system, % recovery was calculated using the concentrations:

% recovery=C₂/C₁×100%.% drug substance loaded was calculated as (100−% recovery.

Loading Time=4.0 hours; Concentration of Methyphenidate Loading Solution=1.0 mg/mL; Volume of Loading Solution=20 mL; Mass of Methylphenidate (maximum load)=1.0 mg/mL×20 mL=20 mg.

TABLE 3

| | | | | MPH | | |
|---|---|---|---|---|---|---|
| | | MPH Loading | MPH Loading | Remaining | | |
| | | Solution Initial | Solution Final | Mass in | MPH Mass | |
| | Sample | Conc. (mg/mL) | Conc. (mg/mL) | Loading | Loaded on | % MPH |
| Resin/pH | Set | Before Loading | After Loading | Solution (mg) | Resin (mg) | Loaded |
| CG-50 (type 1) | 1 | 1.0 | 0.2826 | 5.66 | 14.34 | 71.7 |
| pH 6.0 | 2 | 1.0 | 0.2807 | 5.62 | 14.38 | 71.9 |
| IRP-69 | 1 | 1.0 | 0.0626 | 1.26 | 18.74 | 93.7 |
| pH 6.0 | 2 | 1.0 | 0.0623 | 1.24 | 18.76 | 93.8 |
| 50WX2-100 | 1 | 1.0 | 0.0629 | 1.26 | 18.74 | 93.7 |
| pH 6.0 | 2 | 1.0 | 0.0618 | 1.24 | 18.76 | 93.8 |
| 50WX2-400 | 1 | 1.0 | 0.0662 | 1.32 | 18.68 | 93.4 |
| pH 6.0 | 2 | 1.0 | 0.0655 | 1.32 | 18.68 | 93.4 |
| 50WX4-100 | 1 | 1.0 | 0.0502 | 1.00 | 19.00 | 95.0 |
| pH 6.0 | 2 | 1.0 | 0.0506 | 1.02 | 18.98 | 94.9 |
| 50WX4-400 | 1 | 1.0 | 0.0559 | 1.12 | 18.88 | 94.4 |
| pH 6.0 | 2 | 1.0 | 0.0553 | 1.10 | 18.90 | 94.5 |
| CG-50 (type 1) | 1 | 1.0 | 0.4323 | 8.64 | 11.36 | 56.8 |
| pH 7.0 | 2 | 1.0 | 0.4278 | 8.56 | 11.44 | 57.2 |
| IRP-69 | 1 | 1.0 | 0.0817 | 1.64 | 18.36 | 91.8 |
| pH 7.0 | 2 | 1.0 | 0.0808 | 1.62 | 18.38 | 91.9 |
| 50WX2-100 | 1 | 1.0 | 0.0804 | 1.60 | 18.40 | 92.0 |
| pH 7.0 | 2 | 1.0 | 0.0782 | 1.56 | 18.44 | 92.2 |
| 50WX2-400 | 1 | 1.0 | 0.0838 | 1.68 | 18.32 | 91.6 |
| pH 7.0 | 2 | 1.0 | 0.0829 | 1.66 | 18.34 | 91.7 |
| 50WX4-100 | 1 | 1.0 | 0.0668 | 1.34 | 18.66 | 93.3 |
| pH 7.0 | 2 | 1.0 | 0.0666 | 1.67 | 18.33 | 93.3 |
| 50WX4-400 | 1 | 1.0 | 0.0707 | 1.42 | 18.58 | 92.9 |
| pH 7.0 | 2 | 1.0 | 0.0728 | 1.46 | 18.54 | 92.7 |

(Duplicate Sets of Samples for Each Resin/pH)

Depending on which resin and pH were used, a range of loading was achieved, from about 14 to 19 mg (out of 20 mg), or about 70 to 9500. The resin load samples were prepared in duplicate. The percent drug loading was repeatable. The results demonstrated the feasibility of methylphenidate drug loading using specific resins and loading conditions. Of the six AMBERLITE resins evaluated, up to about 95% of methylphenidate was loaded onto the resins, depending which resin and pH were used. Results for duplicate sets of resin loading samples indicate a repeatable process.

Using the two duplicate sets of samples, one set was used to evaluate methylphenidate release using simulated gastric fluid (SGF) and one set was used to release (extract) methylphenidate using acetone.

The release of methylphenidate from the loaded resins using simulated gastric fluid (SGF) was evaluated. SGF was used to extract methylphenidate from each type of resin. Extraction of methylphenidate was evaluated in a simplified manner using disposable syringes to contain and soak the loaded resins in SGF. The SGF was warmed to 37° C. (body temperature) immediately prior to use but the solution was allowed to cool to ambient temperature during the extraction. No agitation was used.

Following extraction, each sample (˜20 mL SGF and ˜ 200 mg resin) was filtered through a small cotton plug in the syringe tip and collected in a glass vial. For each successive time point, the release (extraction) was performed and the concentration of methylphenidate in SGF was measured by HPLC after 30 minutes, after 3 hours and after 3 more hours, for a total of 3 additive time points at 0.5, 3.5 and 6.5 total hours. The methylphenidate concentration following extraction with SGF was converted to mass (mg) recovered, which was then compared against the mass loaded on the resins, and reported as % recovered after 30 minutes, 3.5 hours, 6.5 hours and total % recovered (after 6.5 hours).

TABLE 4
Release of Methylphenidate from Loaded Resins Using SGF

| | | mg MPH | mg MPH | mg MPH | Total mg | |
|---|---|---|---|---|---|---|
| | mg MPH | Recovered | Recovered | Recovered | MPH | % MPH |
| Resin/pH | Loaded | After 30 minutes | After 3.5 hours | After 6.5 hours | Recovered | Released |
| CG-50 (type 1) | 14.34 | 10.48 | 2.70 | 0.65 | 13.83 | 96.4 |
| pH 6.0 | | | | | | |
| IRP-69 | 18.74 | 1.51 | 1.16 | 1.04 | 3.71 | 19.8 |
| pH 6.0 | | | | | | |
| 50WX2-100 | 18.74 | 0.97 | 1.17 | 0.64 | 2.78 | 14.8 |
| pH 6.0 | | | | | | |
| 50WX2-400 | 18.68 | 1.99 | 1.66 | 1.49 | 5.14 | 27.5 |
| pH 6.0 | | | | | | |
| 50WX4-100 | 19.00 | 0.68 | 0.58 | 0.82 | 2.08 | 10.9 |
| pH 6.0 | | | | | | |
| 50WX4-400 | 18.88 | 1.47 | 1.24 | 1.13 | 3.84 | 20.3 |
| pH 6.0 | | | | | | |
| IRP-69 | 18.36 | 1.78 | 1.36 | 1.21 | 4.35 | 23.7 |
| pH 7.0 | | | | | | |
| 50WX2-100 | 18.40 | 2.14 | 1.04 | 0.89 | 4.07 | 22.1 |
| pH 7.0 | | | | | | |
| 50WX2-400 | 18.32 | 2.25 | 1.91 | 1.68 | 5.84 | 31.9 |
| pH 7.0 | | | | | | |
| 50WX4-100 | 18.66 | 0.83 | 0.77 | 0.69 | 2.29 | 12.3 |
| pH 7.0 | | | | | | |
| 50WX4-400 | 18.58 | 1.70 | 1.44 | 1.26 | 4.40 | 23.7 |
| pH 7.0 | | | | | | |

### Release of Methylphenidate Using Aceton

The extraction of methylphenidate was also performed but using acetone instead of SGF. The amount of methylphenidate released (extracted) from the resins using acetone was generally much lower than with SGF. This indicates that acetone does not facilitate extraction of methylphenidate from the resins and cannot be used to overcome the drug binding of the resins.

TABLE 5
Release of Methylphenidate from Loaded Resins Using Acetone

| | | mg MPH | mg MPH | mg MPH | Total mg | |
|---|---|---|---|---|---|---|
| | mg MPH | Recovered | Recovered | Recovered | MPH | % MPH |
| Resin/pH | Loaded | After 30 minutes | After 3.5 hours | After 6.5 hours | Recovered | Released |
| CG-50 (type 1) | 14.38 | 1.36 | 1.24 | 1.27 | 3.87 | 26.9 |
| pH 6.0 | | | | | | |
| IRP-69 | 18.76 | 0.08 | 0.05 | 0.04 | 0.17 | 0.9 |
| pH 6.0 | | | | | | |
| 50WX2-100 | 18.76 | 0.34 | 0.03 | 0.02 | 0.39 | 2.1 |
| pH 6.0 | | | | | | |
| 50WX2-400 | 18.68 | 0.47 | 0.04 | 0.03 | 0.54 | 2.9 |
| pH 6.0 | | | | | | |
| 50WX4-100 | 18.98 | 0.13 | 0.03 | 0.01 | 0.17 | 0.9 |
| pH 6.0 | | | | | | |
| 50WX4-400 | 18.90 | 0.13 | 0.04 | 0.03 | 0.20 | 1.1 |
| pH 6.0 | | | | | | |
| CG-50 (type 1) | 11.44 | 0.95 | 0.34 | 0.12 | 1.41 | 12.3 |
| pH 7.0 | | | | | | |
| IRP-69 | 18.38 | 0.40 | 0.66 | 0.04 | 1.10 | 6.0 |
| pH 7.0 | | | | | | |
| 50WX2-100 | 18.44 | 0.86 | 0.08 | 0.03 | 0.97 | 5.3 |
| pH 7.0 | | | | | | |
| 50WX2-400 | 18.34 | 0.51 | 0.06 | 0.03 | 0.60 | 3.3 |
| pH 7.0 | | | | | | |
| 50WX4-100 | 18.66 | 0.25 | 0.04 | 0.02 | 0.31 | 1.7 |
| pH 7.0 | | | | | | |
| 50WX4-400 | 18.54 | 0.31 | 0.03 | 0.03 | 0.37 | 2.0 |
| pH 7.0 | | | | | | |

### Example 4. Naltrexone Loading and Release Using AMBERLITE Resins

This study demonstrated the feasibility of naltrexone drug loading using some specific resins and loading conditions. Resin loading for naltrexone was examined. Six types of AMBERLITE resins were selected. Naltrexone drug loading was determined separately at pH 7.0 and pH 8.0 after about 4 hours of resin (˜200 mg) soaking in a solution of naltrexone (1.0 mg/mL×20 mL) in aqueous buffer. An earlier loading study at pH 7-9 indicated that highest loading was at pH 7-8, and longer loading time, up to 48 hours, did not result in significantly more loading.

The method for loading of naltrexone onto the resins was the same as the method used in Example 3. At the resin loading step in the method 200±2 mg of naltrexone was added to 200 mL volumetric flask.

Of the six AMBERLITE resins evaluated, up to about 90% of naltrexone was bound (loaded) onto the resins, depending which resin and pH were used. The loaded resin samples were prepared in duplicate. The repeatable results for duplicate sets of resin loading samples indicate a repeatable process.

Loading Time=4.0 hours; Concentration of Naltrexone Loading Solution=1.0 mg/mL; Volume of Loading Solution=20 mL; Mass of Naltrexone (maximum load)=1.0 mg/mL×20 mL=20 mg.

TABLE 6
Naltrexone Resin Loading Results (Duplicate Sets of Samples for Each Resin/pH)

| | | NTX Loading | NTX Loading | | | |
|---|---|---|---|---|---|---|
| | | Solution Initial | Solution Final | NTX Remaining | NTX Mass | |
| | Sample | Conc. (mg/mL) | Conc. (mg/mL) | Mass in Loading | Loaded on | % NTX |
| Resin/pH | Set | Before Loading | After Loading | Solution (mg) | Resin (mg) | Loaded |
| CG-50 (type 1) | 1 | 1.0 | 0.4669 | 9.34 | 10.66 | 53.3 |
| pH 7.0 | 2 | 1.0 | 0.4704 | 9.40 | 10.60 | 53.0 |
| IRP-69 | 1 | 1.0 | 0.1404 | 2.80 | 17.2 | 86.0 |
| pH 7.0 | 2 | 1.0 | 0.1414 | 2.82 | 17.18 | 85.9 |
| 50WX2-100 | 1 | 1.0 | 0.1024 | 2.04 | 17.96 | 89.8 |
| pH 7.0 | 2 | 1.0 | 0.1026 | 2.06 | 17.94 | 89.7 |
| 50WX2-400 | 1 | 1.0 | 0.1038 | 2.08 | 17.92 | 89.6 |
| pH 7.0 | 2 | 1.0 | 0.1041 | 2.08 | 17.92 | 89.6 |
| 50WX4-100 | 1 | 1.0 | 0.0937 | 1.88 | 18.12 | 90.6 |
| pH 7.0 | 2 | 1.0 | 0.0926 | 1.86 | 18.14 | 90.7 |
| 50WX4-400 | 1 | 1.0 | 0.1081 | 2.16 | 17.84 | 89.2 |
| pH 7.0 | 2 | 1.0 | 0.1068 | 2.14 | 17.86 | 89.3 |
| CG-50 (type 1) | 1 | 1.0 | 0.6026 | 12.06 | 7.94 | 39.7 |
| pH 8.0 | 2 | 1.0 | 0.6063 | 12.22 | 7.88 | 39.4 |
| IRP-69 | 1 | 1.0 | 0.1853 | 3.70 | 16.3 | 81.5 |
| pH 8.0 | 2 | 1.0 | 0.1843 | 3.68 | 16.32 | 81.6 |
| 50WX2-100 | 1 | 1.0 | 0.1485 | 2.98 | 17.02 | 85.1 |
| pH 8.0 | 2 | 1.0 | 0.1479 | 2.96 | 17.04 | 85.2 |
| 50WX2-400 | 1 | 1.0 | 0.1477 | 2.96 | 17.04 | 85.2 |
| pH 8.0 | 2 | 1.0 | 0.1468 | 2.94 | 17.06 | 85.3 |
| 50WX4-100 | 1 | 1.0 | 0.1240 | 2.48 | 17.52 | 87.6 |
| pH 8.0 | 2 | 1.0 | 0.1275 | 2.54 | 17.46 | 87.3 |
| 50WX4-400 | 1 | 1.0 | 0.1532 | 3.06 | 16.94 | 84.7 |
| pH 8.0 | 2 | 1.0 | 0.1525 | 3.06 | 16.94 | 84.7 |

Using the two duplicate sets of samples, one set was used to evaluate naltrexone release using SGF and one set was used to release (extract) naltrexone using acetone.

Release of Naltrexone Using SGF: Release of naltrexone from the loaded resins using SGF was evaluated using the same procedure described for releasing methylphenidate using SGF in Example 3. Six AMBERLITE resins were evaluated.

TABLE 7
Release of Naltrexone from Loaded Resins Using SGF

| | | mg NTX | mg NTX | mg NTX | Total mg | |
|---|---|---|---|---|---|---|
| | mg NTX | Recovered | Recovered | Recovered | NTX | % NTX |
| Resin/pH | Loaded | After 30 minutes | After 3.5 hours | After 6.5 hours | Recovered | Released |
| CG-50 (type 1) | 10.66 | 7.24 | 3.32 | 0.86 | 11.42 | 107.1 |
| pH 7.0 | | | | | | |
| IRP-69 | 17.20 | 2.89 | 2.17 | 1.67 | 6.73 | 39.1 |
| pH 7.0 | | | | | | |
| 50WX2-100 | 17.96 | 1.22 | 1.09 | 1.03 | 3.34 | 18.6 |
| pH 7.0 | | | | | | |
| 50WX2-400 | 17.92 | 2.59 | 2.09 | 1.81 | 6.49 | 36.2 |
| pH 7.0 | | | | | | |
| 50WX4-100 | 18.12 | 0.86 | 0.78 | 0.72 | 2.36 | 13.0 |
| pH 7.0 | | | | | | |
| 50WX4-400 | 17.84 | 2.31 | 1.77 | 1.59 | 5.67 | 31.8 |
| pH 7.0 | | | | | | |
| CG-50 (type 1) | 7.94 | 2.60 | 2.82 | 0.61 | 6.03 | 75.9 |
| pH 8.0 | | | | | | |
| IRP-69 | 16.30 | 2.78 | 1.98 | 1.70 | 6.46 | 39.6 |
| pH 8.0 | | | | | | |
| 50WX2-100 | 17.02 | 1.09 | 0.99 | 0.95 | 3.03 | 17.8 |
| pH 8.0 | | | | | | |
| 50WX2-400 | 17.04 | 2.47 | 2.08 | 1.79 | 6.34 | 37.2 |
| pH 8.0 | | | | | | |
| 50WX4-100 | 17.52 | 0.84 | 0.81 | 1.08 | 2.73 | 15.6 |
| pH 8.0 | | | | | | |
| 50WX4-400 | 16.94 | 2.22 | 1.63 | 1.60 | 5.45 | 32.2 |
| pH 8.0 | | | | | | |

Release of Naltrexone Using Acetone

Release of naltrexone from the loaded resins using acetone was evaluated using the same procedure described for releasing methylphenidate using acetone in Example 3. The amount of naltrexone released (extracted) from the resins using acetone was generally much lower than with SGF. This indicates that acetone does not facilitate extraction of naltrexone from the resins and cannot be used to overcome the drug binding of the resins.

TABLE 8
Recovery of Naltrexone from Loaded Resins Using Acetone

| | | mg NTX | mg NTX | mg NTX | Total mg | |
|---|---|---|---|---|---|---|
| | mg NTX | Recovered | Recovered | Recovered | NTX | % NTX |
| Resin/pH | Loaded | After 30 minutes | After 3.5 hours | After 6.5 hours | Recovered | Released |
| CG-50 (type 1) | 10.60 | 0.48 | 0.03 | 0.0 | 0.51 | 4.8 |
| pH 7.0 | | | | | | |
| IRP-69 | 17.18 | 0.17 | 0.01 | 0.0 | 0.18 | 1.0 |
| pH 7.0 | | | | | | |
| 50WX2-100 | 17.94 | 1.02 | 0.09 | 0.0 | 1.11 | 6.2 |
| pH 7.0 | | | | | | |
| 50WX2-400 | 17.92 | 1.59 | 0.07 | 0.0 | 1.66 | 9.3 |
| pH 7.0 | | | | | | |
| 50WX4-100 | 18.14 | 0.32 | 0.03 | 0.0 | 0.35 | 1.9 |
| pH 7.0 | | | | | | |
| 50WX4-400 | 17.86 | 0.44 | 0.02 | 0.0 | 0.46 | 2.6 |
| pH 7.0 | | | | | | |
| CG-50 (type 1) | 7.88 | 0.30 | 0.01 | 0.0 | 0.31 | 3.9 |
| pH 8.0 | | | | | | |
| IRP-69 | 16.32 | 0.30 | 0.01 | 0.0 | 0.31 | 1.9 |
| pH 8.0 | | | | | | |
| 50WX2-100 | 17.04 | 1.48 | 0.10 | 0.0 | 1.49 | 8.7 |
| pH 8.0 | | | | | | |
| 50WX2-400 | 17.06 | 2.37 | 0.09 | 0.0 | 2.46 | 14.4 |
| pH 8.0 | | | | | | |
| 50WX4-100 | 17.46 | 0.33 | 0.05 | 0.0 | 0.38 | 2.2 |
| pH 8.0 | | | | | | |
| 50WX4-400 | 16.94 | 0.28 | 0.02 | 0.0 | 0.30 | 1.8 |
| pH 8.0 | | | | | | |

### Example 5. Further Studies of Loading and Release of Methylphenidate and Naltrexone

Based on experimental data in Examples 3 and 4, six more resins were identified for evaluation. The same six resins previously evaluated plus six additional resins for a total of twelve were all evaluated. All twelve were evaluated for their capacity to load naltrexone and methylphenidate at two preselected pH values, and their capacity to release methylphenidate and naltrexone in SGF within 1 hour.

Naltrexone was loaded at pH 7.0 and 8.0. Methylphenidate was loaded at pH 5.0 and pH 6.0.

Calculated:mg/mL analyte=peak area sample/peak area reference solution=mg/mL reference solution.

Calculated:mg Released=mg/mL analyte in sample×20 mL(SGF)

Calculated:% Released=mg Released(in SGF release solution)/mg Loaded(in resin)×100.

TABLE 9
Release of Naltrexone from Loaded
Resins Using SGF After 1 Hour

| | mg NTX | mg NTX | % NTX |
|---|---|---|---|
| Resin/pH | Loaded | Recovered | Released |
| CG-50 (type 1) | 10.34 | 10.62 | 102.7 |
| pH 7.0 | | | |
| IRP-69 | 17.20 | 3.23 | 18.8 |
| pH 7.0 | | | |
| 50WX2-100 | 17.98 | 3.08 | 17.1 |
| pH 7.0 | | | |
| 50WX2-400 | 17.94 | 3.11 | 17.3 |
| pH 7.0 | | | |
| 50WX4-100 | 17.80 | 2.72 | 15.3 |
| pH 7.0 | | | |
| 50WX4-400 | 18.34 | 2.69 | 14.7 |
| pH 7.0 | | | |
| HP2MGL | 7.78 | 6.24 | 80.2 |
| pH 7.0 | | | |
| WK10 | 10.98 | 9.09 | 82.8 |
| pH 7.0 | | | |
| WK11 | 13.14 | 12.52 | 95.3 |
| pH 7.0 | | | |
| WK40L | 10.52 | 10.39 | 98.8 |
| pH 7.0 | | | |
| WK100 | 13.44 | 13.32 | 99.1 |
| pH 7.0 | | | |
| WT01S | 12.74 | 12.76 | 100.2 |
| pH 7.0 | | | |
| CG-50 (type 1) | 8.12 | 8.50 | 104.7 |
| pH 8.0 | | | |
| IRP-69 | 16.42 | 3.12 | 19.0 |
| pH 8.0 | | | |
| 50WX2-100 | 17.16 | 2.96 | 17.2 |
| pH 8.0 | | | |
| 50WX2-400 | 17.14 | 3.02 | 17.6 |
| pH 8.0 | | | |
| 50WX4-100 | 17.32 | 2.67 | 15.4 |
| pH 8.0 | | | |
| 50WX4-400 | 18.00 | 2.61 | 14.5 |
| pH 8.0 | | | |
| HP2MGL | 12.72 | 11.00 | 86.5 |
| pH 8.0 | | | |
| WK10 | 9.76 | 8.37 | 85.8 |
| pH 8.0 | | | |
| WK11 | 12.20 | 11.89 | 97.5 |
| pH 8.0 | | | |
| WK40L | 9.44 | 9.37 | 99.3 |
| pH 8.0 | | | |
| WK100 | 12.16 | 12.31 | 101.2 |
| pH 8.0 | | | |
| WT01S | 11.3 | 11.34 | 100.4 |
| pH 8.0 | | | |

TABLE 10
Release of Methylphenidate from Loaded
Resins Using SGF After 1 Hour

| | mg MPH | mg MPH | % MPH |
|---|---|---|---|
| Resin/pH | Loaded | Recovered | Released |
| CG-50 (type 1) | 14.56 | 13.43 | 92.2 |
| pH 5.0 | | | |
| IRP-69 | 18.88 | 1.69 | 9.0 |
| pH 5.0 | | | |
| 50WX2-100 | 18.82 | 2.16 | 11.5 |
| pH 5.0 | | | |
| 50WX2-400 | 18.76 | 2.26 | 12.0 |
| pH 5.0 | | | |
| 50WX4-100 | 18.94 | 1.75 | 9.2 |
| pH 5.0 | | | |
| 50WX4-400 | 19.22 | 1.69 | 8.8 |
| pH 5.0 | | | |
| HP2MGL | 2.64 | 2.24 | 84.8 |
| pH 5.0 | | | |
| WK10 | 16.40 | 13.85 | 84.5 |
| pH 5.0 | | | |
| WK11 | 14.94 | 12.99 | 86.9 |
| pH 5.0 | | | |
| WK40L | 12.10 | 11.02 | 91.1 |
| pH 5.0 | | | |
| WK100 | 14.90 | 13.30 | 89.3 |
| pH 5.0 | | | |
| WT10S | 15.76 | 14.22 | 90.2 |
| pH 5.0 | | | |
| CG-50 (type 1) | 14.08 | 10.05 | 71.4 |
| pH 6.0 | | | |
| IRP-69 | 18.70 | 1.72 | 9.2 |
| pH 6.0 | | | |
| 50WX2-100 | 18.72 | 2.16 | 11.5 |
| pH 6.0 | | | |
| 50WX2-400 | 18.66 | 2.22 | 11.9 |
| pH 6.0 | | | |
| 50WX4-100 | 18.90 | 1.74 | 9.2 |
| pH 6.0 | | | |
| 50WX4-400 | 19.10 | 1.67 | 8.7 |
| pH 6.0 | | | |
| HP2MGL | 2.84 | 2.61 | 91.9 |
| pH 6.0 | | | |
| WK10 | 16.04 | 13.63 | 85.0 |
| pH 6.0 | | | |
| WK11 | 15.02 | 11.68 | 77.8 |
| pH 6.0 | | | |
| WK40L | 11.76 | 10.97 | 93.3 |
| pH 6.0 | | | |
| WK100 | 15.08 | 13.76 | 91.2 |
| pH 6.0 | | | |
| WT01S | 14.94 | 14.04 | 94.0 |
| pH 6.0 | | | |

### Example 6: Release of Naltrexone and Methylphenidate in Water, Acetone and Acetonitrile

To evaluate abuse potential, release (extraction) of each drug was examined separately using pure water (i.e., no pH adjustment), acetone and acetonitrile. The seven resins were used, based on their capacity to load and release either naltrexone or methylphenidate using SGF. The seven resins were selected on the basis of loading and releasing at least 10 mg (500 of potential 20 mg load) of either naltrexone or methylphenidate within 1 hour using SGF with constant mixing at 37° C.

The same method used in the SGF release was also used for release with water, acetone or acetonitrile. A 1-hour time point was used for direct comparison with SGF release. A 24-hour time point was added to examine the possibility of slow release.

TABLE 11
Release of Naltrexone from Loaded Resins After 1 Hour using Water, Acetone or Acetonitrile

| | Water | | | Acetone | | | Acetonitrile | | |
|---|---|---|---|---|---|---|---|---|---|
| | mg | mg | % | mg | mg | % | mg | mg | % |
| | NTX | NTX | NTX | NTX | NTX | NTX | NTX | NTX | NTX |
| Resin/pH | Loaded | Recovered | Released | Loaded | Recovered | Released | Loaded | Recovered | Released |
| HP2MGL | 7.70 | 0.83 | 10.8 | 7.54 | 8.18 | 108.5 | 7.76 | 8.06 | 103.9 |
| pH 7.0 | | | | | | | | | |
| WK10 | 11.44 | 0.12 | 1.0 | 11.26 | 1.21 | 10.7 | 11.04 | 1.29 | 11.7 |
| pH 7.0 | | | | | | | | | |
| WK11 | 13.34 | 0.04 | 0.3 | 13.88 | 6.48 | 46.7 | 13.70 | 1.02 | 7.4 |
| pH 7.0 | | | | | | | | | |
| WK40L | 10.46 | 0.08 | 0.8 | 10.52 | 0.28 | 2.7 | 11.14 | 0.27 | 2.4 |
| pH 7.0 | | | | | | | | | |
| WK100 | 13.64 | 0.09 | 0.7 | 13.40 | 8.63 | 64.4 | 13.50 | 2.13 | 15.8 |
| pH 7.0 | | | | | | | | | |
| WT01S | 12.92 | 0.12 | 0.9 | 12.70 | 9.25 | 72.8 | 12.60 | 2.27 | 18.0 |
| pH 7.0 | | | | | | | | | |
| CG50 | 9.94 | 0.10 | 1.0 | 10.56 | 0.96 | 9.1 | 10.34 | 0.63 | 6.1 |
| pH 7.0 | | | | | | | | | |
| HP2MGL | 12.88 | 0.80 | 6.2 | 12.76 | 12.92 | 101.3 | 12.82 | 13.18 | 102.8 |
| pH 8.0 | | | | | | | | | |
| WK10 | 9.68 | 0.17 | 1.8 | 9.52 | 1.17 | 12.3 | 10.78 | 1.48 | 13.7 |
| pH 8.0 | | | | | | | | | |
| WK11 | 12.56 | 0.10 | 0.8 | 13.06 | 2.15 | 16.5 | 12.98 | 1.22 | 9.4 |
| pH 8.0 | | | | | | | | | |
| WK40L | 9.10 | 0.14 | 1.5 | 9.10 | 0.18 | 2.0 | 9.06 | 0.25 | 2.8 |
| pH 8.0 | | | | | | | | | |
| WK100 | 12.18 | 0.12 | 1.0 | 12.06 | 7.61 | 63.1 | 12.34 | 2.78 | 22.5 |
| pH 8.0 | | | | | | | | | |
| WT01S | 10.94 | 0.33 | 3.0 | 10.92 | 5.08 | 46.5 | 10.72 | 2.45 | 22.9 |
| pH 8.0 | | | | | | | | | |
| CG50 | 7.92 | 0.22 | 2.8 | 8.24 | 0.84 | 10.2 | 8.04 | 0.80 | 10.0 |
| pH 8.0 | | | | | | | | | |

TABLE 12
Release of Naltrexone from Loaded Resins After 24 Hours using Water, Acetone or Acetonitrile

| | Water | | | Acetone | | | Acetonitrile | | |
|---|---|---|---|---|---|---|---|---|---|
| | mg | mg | % | mg | mg | % | mg | mg | % |
| | NTX | NTX | NTX | NTX | NTX | NTX | NTX | NTX | NTX |
| Resin/pH | Loaded | Recovered | Released | Loaded | Recovered | Released | Loaded | Recovered | Released |
| HP2MGL | 7.70 | 2.04 | 26.5 | 7.54 | 8.35 | 110.7 | 7.76 | 8.25 | 106.3 |
| pH 7.0 | | | | | | | | | |
| WK10 | 11.44 | 0.17 | 1.5 | 11.26 | 1.31 | 11.6 | 11.04 | 1.31 | 11.9 |
| pH 7.0 | | | | | | | | | |
| WK11 | 13.34 | 0.07 | 0.5 | 13.88 | 10.54 | 15.2 | 13.70 | 1.45 | 10.6 |
| pH 7.0 | | | | | | | | | |
| WK40L | 10.46 | 0.13 | 1.2 | 10.52 | 0.30 | 2.9 | 11.14 | 0.27 | 2.4 |
| pH 7.0 | | | | | | | | | |
| WK100 | 13.64 | 0.16 | 1.2 | 13.40 | 10.04 | 74.9 | 13.50 | 2.78 | 20.6 |
| pH 7.0 | | | | | | | | | |
| WT01S | 12.92 | 0.23 | 1.8 | 12.70 | 10.83 | 85.3 | 12.60 | 2.91 | 23.1 |
| pH 7.0 | | | | | | | | | |
| CG50 | 9.94 | 0.27 | 2.7 | 10.56 | 1.14 | 10.8 | 10.34 | 0.65 | 6.3 |
| pH 7.0 | | | | | | | | | |
| HP2MGL | 12.88 | 2.51 | 19.5 | 12.76 | 13.45 | 105.4 | 12.82 | 13.30 | 103.7 |
| pH 8.0 | | | | | | | | | |
| WK10 | 9.68 | 0.25 | 2.6 | 9.52 | 1.21 | 12.7 | 10.78 | 1.50 | 13.9 |
| pH 8.0 | | | | | | | | | |
| WK11 | 12.56 | 0.12 | 1.0 | 13.06 | 3.79 | 29.0 | 12.98 | 1.16 | 8.9 |
| pH 8.0 | | | | | | | | | |
| WK40L | 9.10 | 0.23 | 2.5 | 9.10 | 0.19 | 2.1 | 9.06 | 0.26 | 2.9 |
| pH 8.0 | | | | | | | | | |
| WK100 | 12.18 | 0.19 | 1.6 | 12.06 | 10.12 | 83.9 | 12.34 | 3.71 | 30.1 |
| pH 8.0 | | | | | | | | | |
| WT01S | 10.94 | 0.43 | 3.9 | 10.92 | 6.21 | 56.9 | 10.72 | 2.63 | 24.5 |
| pH 8.0 | | | | | | | | | |
| CG50 | 7.92 | 0.55 | 6.9 | 8.24 | 0.89 | 10.8 | 8.04 | 0.82 | 10.2 |
| pH 8.0 | | | | | | | | | |

The data in Table 11 and 12 shows no significant amount of naltrexone was differentially released from the resins using water, acetone or acetonitrile, even after 24 hours.

TABLE 13
Release of Methylphenidate (MPH) from Loaded Resins
After 1 Hour using Water, Acetone or Acetonitrile

| | Water | | | Acetone | | | Acetonitrile | | |
|---|---|---|---|---|---|---|---|---|---|
| | mg | mg | % | mg | mg | % | mg | mg | % |
| | MPH | MPH | MPH | MPH | MPH | MPH | MPH | MPH | MPH |
| Resin/pH | Loaded | Recovered | Released | Loaded | Recovered | Released | Loaded | Recovered | Released |
| HP2MGL | 2.52 | 1.32 | 52.4 | 2.38 | 2.67 | 112.2 | 2.14 | 2.43 | 113.6 |
| pH 5.0 | | | | | | | | | |
| WK10 | 15.64 | 0.04 | 0.3 | 15.14 | 2.47 | 16.3 | 14.98 | 0.55 | 3.7 |
| pH 5.0 | | | | | | | | | |
| WK11 | 13.42 | 0.05 | 0.4 | 13.56 | 1.53 | 11.3 | 13.42 | 0.28 | 2.1 |
| pH 5.0 | | | | | | | | | |
| WK40L | 11.42 | 0.05 | 0.4 | 11.34 | 0.26 | 2.3 | 11.70 | 0.16 | 1.4 |
| pH 5.0 | | | | | | | | | |
| WK100 | 13.42 | 0.08 | 0.6 | 13.42 | 1.15 | 8.6 | 13.02 | 0.16 | 1.2 |
| pH 5.0 | | | | | | | | | |
| WT01S | 13.06 | 0.03 | 0.2 | 13.52 | 2.68 | 19.8 | 12.46 | 0.43 | 3.5 |
| pH 5.0 | | | | | | | | | |
| CG50 | 14.02 | 0.02 | 0.1 | 13.98 | 2.54 | 18.2 | 13.94 | 0.51 | 3.7 |
| pH 5.0 | | | | | | | | | |
| HP2MGL | 3.18 | 1.10 | 34.6 | 3.28 | 3.35 | 102.1 | 3.14 | 3.02 | 96.2 |
| pH 6.0 | | | | | | | | | |
| WK10 | 14.68 | 0.04 | 0.3 | 14.80 | 2.49 | 16.8 | 14.76 | 0.50 | 3.4 |
| pH 6.0 | | | | | | | | | |
| WK11 | 14.20 | 0.05 | 0.4 | 14.74 | 2.33 | 15.8 | 14.10 | 0.45 | 3.2 |
| pH 6.0 | | | | | | | | | |
| WK40L | 11.20 | 0.03 | 0.3 | 11.34 | 0.32 | 2.8 | 11.64 | 0.17 | 1.5 |
| pH 6.0 | | | | | | | | | |
| WK100 | 13.82 | 0.03 | 0.2 | 13.40 | 1.56 | 11.6 | 13.42 | 0.26 | 1.9 |
| pH 6.0 | | | | | | | | | |
| WT01S | 13.14 | 0.03 | 0.2 | 13.20 | 3.30 | 25.0 | 12.88 | 0.70 | 5.4 |
| pH 6.0 | | | | | | | | | |
| CG50 | 13.70 | 0.11 | 0.8 | 13.28 | 2.82 | 21.2 | 13.18 | 0.67 | 5.1 |
| pH 6.0 | | | | | | | | | |

TABLE 14
Release of Methylphenidate (MPH) from Loaded Resins
After 24 Hours using Water, Acetone or Acetonitrile

| | Water | | | Acetone | | | Acetonitrile | | |
|---|---|---|---|---|---|---|---|---|---|
| | mg | mg | % | mg | mg | % | mg | mg | % |
| | MPH | MPH | MPH | MPH | MPH | MPH | MPH | MPH | MPH |
| Resin/pH | Loaded | Recovered | Released | Loaded | Recovered | Released | Loaded | Recovered | Released |
| HP2MGL | 2.52 | 1.66 | 65.9 | 2.38 | 2.49 | 104.6 | 2.14 | 2.09 | 97.7 |
| pH 5.0 | | | | | | | | | |
| WK10 | 15.64 | 0.06 | 0.4 | 15.14 | 3.50 | 23.1 | 14.98 | 0.66 | 4.4 |
| pH 5.0 | | | | | | | | | |
| WK11 | 13.42 | 0.05 | 0.4 | 13.56 | 1.38 | 10.2 | 13.42 | 0.24 | 1.8 |
| pH 5.0 | | | | | | | | | |
| WK40L | 11.42 | 0.07 | 0.6 | 11.34 | 0.25 | 2.2 | 11.70 | 0.14 | 1.2 |
| pH 5.0 | | | | | | | | | |
| WK100 | 13.42 | 0.10 | 0.7 | 13.42 | 1.14 | 8.5 | 13.02 | 0.16 | 1.2 |
| pH 5.0 | | | | | | | | | |
| WT01S | 13.06 | 0.06 | 0.5 | 13.52 | 2.72 | 20.1 | 12.46 | 0.47 | 3.8 |
| pH 5.0 | | | | | | | | | |
| CG50 | 14.02 | 0.07 | 0.5 | 13.98 | 3.06 | 21.9 | 13.94 | 0.63 | 4.5 |
| pH 5.0 | | | | | | | | | |
| HP2MGL | 3.18 | 1.74 | 54.7 | 3.28 | 3.30 | 100.6 | 3.14 | 2.83 | 90.1 |
| pH 6.0 | | | | | | | | | |
| WK10 | 14.68 | 0.06 | 0.4 | 14.80 | 4.04 | 27.3 | 14.76 | 0.55 | 3.7 |
| pH 6.0 | | | | | | | | | |
| WK11 | 14.20 | 0.05 | 0.4 | 14.74 | 2.22 | 15.1 | 14.10 | 0.36 | 2.6 |
| pH 6.0 | | | | | | | | | |
| WK40L | 11.20 | 0.09 | 0.8 | 11.34 | 0.30 | 2.6 | 11.64 | 0.14 | 1.2 |
| pH 6.0 | | | | | | | | | |
| WK100 | 13.82 | 0.24 | 1.7 | 13.40 | 1.60 | 11.9 | 13.42 | 0.24 | 1.8 |
| pH 6.0 | | | | | | | | | |
| WT01S | 13.14 | 0.06 | 0.5 | 13.20 | 3.52 | 26.7 | 12.88 | 0.78 | 6.1 |
| pH 6.0 | | | | | | | | | |
| CG50 | 13.70 | 0.06 | 0.4 | 13.28 | 3.59 | 27.0 | 13.18 | 0.98 | 7.4 |
| pH 6.0 | | | | | | | | | |

The data Tables 13 and 14 shows that no significant amount of methylphenidate was differentially released from the resins using water, acetone or acetonitrile, even after 24 hours.

### Example 7: Enhanced Loading and Release of Naltrexone Using SGF

The method for loading methylphenidate and naltrexone was modified to increase the amount of drug loaded to therapeutic dosage levels (>20 mg). This was done by increasing the amount and concentration of drug in the loading solution from 1.0 mg/mL×20 mL to 3.0 mg/mL×10 mL. Drug loading was examined at 4, 12 and 24 hours.

Methods: A 50 mM sodium phosphate monobasic dihydrate pH buffer stock solution was prepared. The pH buffer solution series was used to evaluate AMBERLITE/DOWEX/DIAION resin drug-loading conditions. NaOH was used to adjust pH of the solution to pH=5.0±0.1, pH 6.0±0.1, pH 7.0±0.1, and pH 8.0±0.1.

Preparation of SGF

1. The following were added to a 1 L volumetric flask:

a. 2.0 g NaCl

b. 3.2 g pepsin

c. 7 mL HCl

2. Brought to volume with water
3. Flask was shaken/inverted until contents were dissolved and thoroughly mixed

Results

TABLE 15
Enhanced Methylphenidate Resin Loading and Release Using SGF

| | mg MPH | mg MPH | mg MPH | mg MPH | |
|---|---|---|---|---|---|
| | Loaded | Loaded | Loaded | Recovered | % MPH |
| Resin/pH | (4 hr) | (12 hr) | (24 hr) | (1 hr) | Released |
| HP2MGL | 4.47 | 4.47 | 4.53 | 4.51 | 99.6 |
| pH 5.0 | | | | | |
| WK10 | 24.96 | 25.92 | 26.31 | 22.28 | 84.7 |
| pH 5.0 | | | | | |
| WK11 | 22.26 | 23.46 | 23.70 | 20.69 | 87.3 |
| pH 5.0 | | | | | |
| WK40L | 21.39 | 22.17 | 22.23 | 21.76 | 97.9 |
| pH 5.0 | | | | | |
| WK100 | 24.30 | 24.99 | 25.14 | 22.76 | 90.5 |
| pH 5.0 | | | | | |
| WT01S | 24.15 | 24.69 | 24.81 | 23.33 | 94.0 |
| pH 5.0 | | | | | |
| CG-50 | 22.32 | 23.40 | 23.67 | 22.90 | 96.7 |
| (type 1) | | | | | |
| pH 5.0 | | | | | |
| HP2MGL | 5.34 | 5.46 | 5.58 | 5.29 | 94.8 |
| pH 6.0 | | | | | |
| WK10 | 24.66 | 25.80 | 26.22 | 23.39 | 89.2 |
| pH 6.0 | | | | | |
| WK11 | 24.09 | 25.53 | 25.86 | 23.95 | 92.6 |
| pH 6.0 | | | | | |
| WK40L | 21.18 | 22.05 | 22.14 | 21.94 | 99.1 |
| pH 6.0 | | | | | |
| WK100 | 24.66 | 25.32 | 25.44 | 24.66 | 96.9 |
| pH 6.0 | | | | | |
| WT01S | 22.74 | 24.69 | 25.05 | 24.60 | 98.2 |
| pH 6.0 | | | | | |
| CG-50 | 21.00 | 22.41 | 23.46 | 22.33 | 95.2 |
| (type 1) | | | | | |
| pH 6.0 | | | | | |

Note
* 24 hour time point was used to calculate % MPH released.

TABLE 16
Enhanced Naltrexone Resin Loading and Release Using SGF

| | mg NTX | mg NTX | mg NTX | mg NTX | |
|---|---|---|---|---|---|
| | Loaded | Loaded | Loaded | Recovered | % NTX |
| Resin/pH | (4 hr) | (12 hr) | (24 hr) | (1 hr) | Released |
| HP2MGL | 11.31 | 11.46 | 11.55 | 12.76 | 110.5 |
| pH 7.0 | | | | | |
| WK10 | 19.59 | 21.54 | 22.62 | 20.40 | 90.2 |
| pH 7.0 | | | | | |
| WK11 | 23.10 | 24.78 | 25.17 | 25.61 | 101.7 |
| pH 7.0 | | | | | |
| WK40L | 21.45 | 22.29 | 22.38 | 23.88 | 106.7 |
| pH 7.0 | | | | | |
| WK100 | 24.00 | 24.45 | 24.54 | 26.59 | 108.4 |
| pH 7.0 | | | | | |
| WT01S | 24.12 | 24.57 | 24.63 | 26.16 | 106.2 |
| pH 7.0 | | | | | |
| CG-50 | 20.10 | 20.16 | 20.19 | 22.75 | 112.7 |
| (type 1) | | | | | |
| pH 7.0 | | | | | |
| HP2MGL | 17.7 | 18.03 | 18.12 | 16.99 | 93.8 |
| pH 8.0 | | | | | |
| WK10 | 19.26 | 21.51 | 22.53 | 18.84 | 83.6 |
| pH 8.0 | | | | | |
| WK11 | 22.53 | 24.78 | 25.26 | 24.97 | 98.9 |
| pH 8.0 | | | | | |
| WK40L | 21.03 | 21.90 | 21.99 | 22.61 | 102.8 |
| pH 8.0 | | | | | |
| WK100 | 23.40 | 23.82 | 23.85 | 25.12 | 105.3 |
| pH 8.0 | | | | | |
| WT01S | 23.88 | 24.24 | 24.18 | 24.80 | 102.6 |
| pH 8.0 | | | | | |
| CG-50 | 18.69 | 18.69 | 18.75 | 20.75 | 110.7 |
| (type 1) | | | | | |
| pH 8.0 | | | | | |

Note
* 24 hour time point is used to calculate % NTX released.

As indicated in Tables 15 and 6, resins were mostly loaded after the first 4 hours (typically >90% depending on the resin). Table 15 and Table 16 show the amount of methylphenidate and naltrexone, respectively loaded using different resins after 4, 12 and 24 hours.

Tables 15 and 16 also show the amount (mg) of methylphenidate and naltrexone released using SGF after 1 hour and the percent of the loaded drug released. The data indicates that more than 20 mg of each drug were loaded and released in SGF within 1 hour for most of the resins evaluated. For all the resins, more than 80% of the drugs loaded were released within 1 hour.

### Example 8: Dissolution Release of Methylphenidate and Naltrexone from Resins

Drug release of methylphenidate and naltrexone for different resins were tested is a dissolution apparatus and analyzed by HPLC.

Methods and Results

Resins were screened to evaluate their capacity to load methylphenidate and/or naltrexone. Several resins demonstrated the capacity to ionically bind therapeutic dosages (about 30 mg) of active pharmaceutical ingredient (API) using <250 mg of the resin.

Release in Simulated Gastric Fluid (SGF) was examined.

The loading conditions were selected to evaluate prototypes with a drug-binding resin component. Therapeutic dosage levels of about 30 mg were successfully loaded onto seven different types of resins. Drug loading was optimized only enough to independently and separately load about 30 mg of one API onto 250 mg of resin.

### Resin Loading:

1. Prepared tubes having 4.0 mg/mL loading solution of methylphenidate
2. Weighed 250±2.5 mg portions of dried, activated resins for each test sample and transferred to tube.
3. Transferred 10 mL of buffer to tube
4. Mixed by stirring on a rotational mixer set to 230 rpm at ambient temperature for 4 hours.
5. Separated resins from loading solution by centrifugation and stored loading solution.
6. Repeated Steps 1-8 with naltrexone test samples.

### Dissolution Testing Conditions:

A Dissolution Apparatus (Agilent Model 708-DS) instrument was used to perform the testing using Apparatus 2 (Paddles). Dissolution was performed using Simulated Gastric Fluid (SGF). A total of eight different types of resins were evaluated, each was evaluated in duplicate.

The Dissolution conditions used to determine the release rates of methylphenidate and naltrexone are presented below in Table 17.

TABLE 17
Dissolution Test Conditions and Instrument Parameters

| Parameter | Value or Range | |
|---|---|---|
| Apparatus | USP Apparatus 2 (Paddles) | |
| Rotational Speed for Paddles | 25 | rpm |
| Medium | Simulated Gastric Fluid (SGF) | |
| Volume | 500 | mL |

| | |
|---|---|
| Temperature | 37 ± 0.5° C. |
| Sampling Time Points | 15, 30, 45, and 60 minutes |
| Sample Volume | ≤1 mL unfiltered |

Methylphenidate and naltrexone dissolution rates were evaluated simultaneously (in same vessel and at same time points). The dissolution samples (<1 mL) were collected after 15, 30, 45 and 60 minutes and analyzed by HPLC. Drug release samples for methylphenidate and naltrexone were tested concurrently using the same test method and same HPLC injection.

### Analysis of Dissolution Samples by HPLC

Solutions:
Loading Buffer: 50 mM Sodium Phosphate Monobasic Dihydrate, pH adjusted to 5.00.1 for Methylphenidate and 7.0±0.1 for Naltrexone
Methylphenidate Resin Loading Solution: 4.0 mg/mL MPH in pH 5.0 Loading Buffer
Naltrexone Resin Loading Solution: 4.0 mg/mL NTX in pH 7.0 Loading Buffer
Mobile Phase A: Phosphate buffer—HPLC-grade water with 20 mM Sodium Phosphate Monobasic Dihydrate, pH adjusted to 2.3±0.1 with phosphoric acid
Mobile Phase B: 1000 m Acetonitrile
Mobile Phase C: (System Wash Solvent): Purified Water, PLC grade minimum

The chromatographic conditions and HPLC parameters are listed below in Table 18.

TABLE 18
Chromatographic Conditions and Instrument Parameter

| Parameter | Range | |
|---|---|---|
| Instrument | HPLC system with gradient pump, | |
| | column heater and UV detector at 210 nm | |
| | A | B |
| Mobile Phase | 20 mM Phosphate Buffer, pH | Acetonitrile |
| Composition | adjusted to 2.3 | |

| Flow Rate | 1.0 mL/min | | |
|---|---|---|---|
| Column | Thermo Scientific Hypersil Gold C8 | | |
| | RP 150 × 4.6 mm, 3 μm | | |
| Guard Column | N/A | | |
| Column Temperature | 40° C. | | |
| | Time | % A | % B |
| Gradient Table | 0.00 | 95 | 5 |
| | 1.00 | 95 | 5 |
| | 6.00 | 75 | 25 |
| | 10.00 | 75 | 25 |
| | 11.00 | 95 | 5 |
| | 18.00 | 95 | 5 |

| | |
|---|---|
| Runtime | 18.00 min |
| Injection volume | 1.0 μL |
| Detection Wavelength | 210 nm |
| Needle and Seal Rinse | 90:10 H₂O:Acetonitrile |
| Solution | |

### Preparation of Mobile Phase and Diluent:

Mobile Phase a (20 mM Phosphate Buffer, pH=2.3):

Weighed about 3.120 g Sodium Phosphate Monobasic Dihydrate and transferred to a clean, 1-L mobile phase container. Added 1000 mL Purified Water. Dissolved and mixed by stirring. Adjusted buffer pH to 2.3±0.1 with phosphoric acid. Used dilute sodium hydroxide to back-adjust pH.

Mobile Phase B: 100% acetonitrile

Mobile Phase C: 100% water

Diluent: 50 mM sodium phosphate, with pH adjusted to 5.0 for Methylphenidate and 7.0 for Naltrexone using 1N NaOH.

### Standard Preparation:

Prepared working standards of naltrexone and methylphenidate separately at a concentration of about 2.0 mg/mL. Dilute 4.0 mg/mL Loading Solution 1:1 with Mobile Phase A.

### HPLC Method:

1. Flow rate was set to 1.0 mL/min with Mobile Phase composition of 5% B:95% Mobile Phase C for at least 10 min.
2. Equilibrated column temperature at 40° C. using initial mobile phase gradient conditions (95% A:5% B) for at least 10 minutes. Monitored baseline at 210 nm until a stable baseline is achieved.
3. One or more sample test injections were made to equilibrate the column and verify typical chromatography prior to initiating the sequence.
4. Performed the injection sequence in Table 19.

TABLE 19

| Step Number | Solution | No. of Injections |
|---|---|---|
| 1 | Diluent Blank | 1-2 |
| 2 | Working Standard | 3 |
| 3 | Media (SGF) Blank | 1-2 |
| 4 | Sample Preparations (up to 6) | 1 |
| 5 | Working Standard | 1 |
| 6 | Sample Preparations (up to 6) | |
| 7 | Working Standard | 1 |

Determined amount naltrexone or methylphenidate loaded by HPLC using 2.0 mg/mL reference standard.

$$Calculated \text{:} mg \text{/} mL naltrexone ( or methylphenidate ) recovered = \\ ( \frac{peak area sample}{peak area reference solution} ) \times concentration reference solution$$

$$Calculated \text{:} \% recovered = ( \frac{\frac{mg}{mL} recovered}{concentration loading solution} ) \times 100$$

$$Calculated \text{:} \% loaded = 100 \% - \% recovered$$

$$Calculated \text{:} mg naltrexone ( or methylphenidate ) loaded = Amount of loading solution \times concentration of loading solution \times \% loaded$$

### Determination of Release Rates (Dissolution Profiles)

Dissolution release sample concentrations were determined by HPLC versus external reference standard solutions of known concentrations. The determined sample concentrations were then used to calculate the amount (mg) of drug released in the dissolution vessels at each time point. The amount (mg) released is divided by the amount (mg) loaded (×100) to determine the percent released at each sample time point. The percent of each drug released was plotted against time to determine the release rates (dissolution profiles).

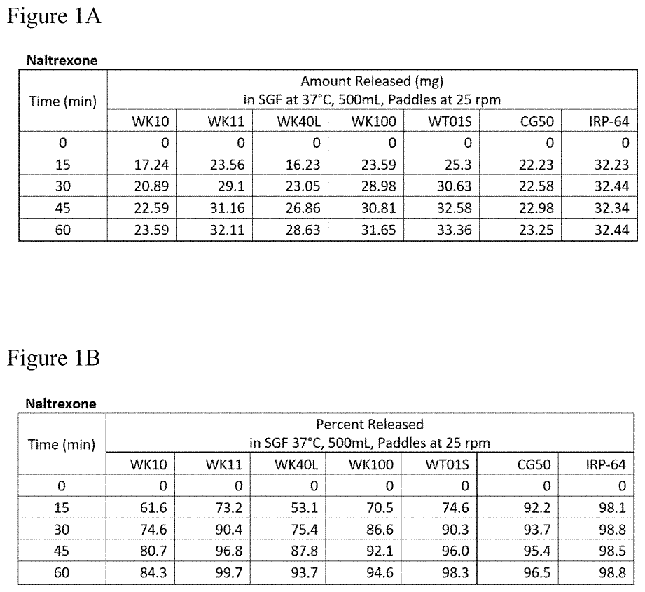

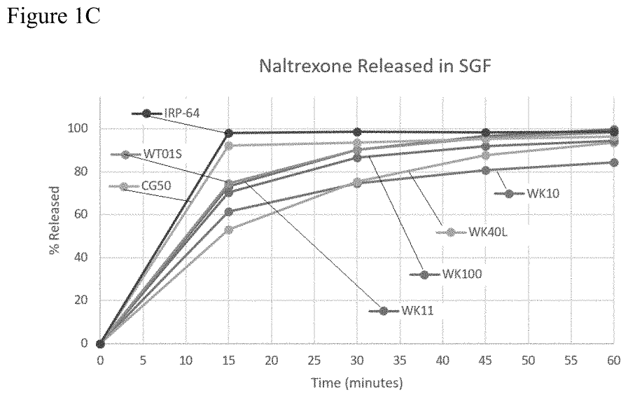

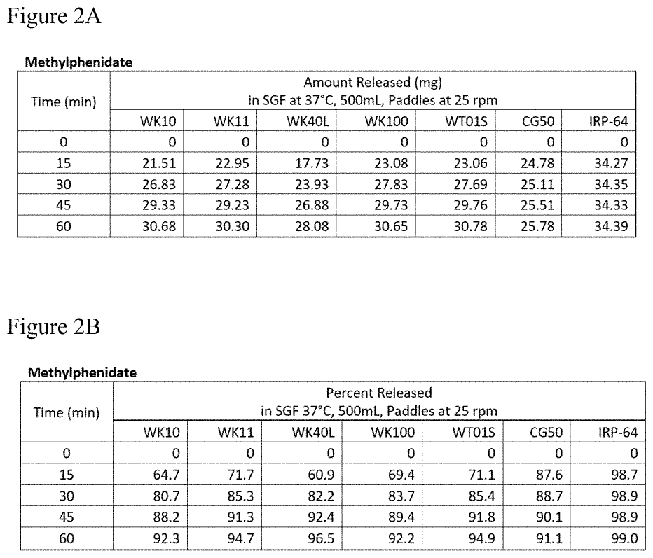

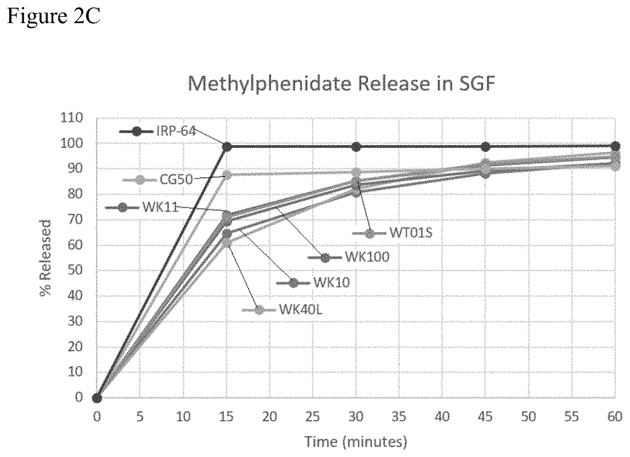

Seven different resins were found to have potentially acceptable drug loading and release (in SGF) properties for one or both drugs. For those seven resins, greater than 80% of both drugs were released within 45 minutes, and virtually all drug substance was released within 60 minutes. The results for dissolution release of naltrexone and methylphenidate are presented in FIGS. 1A-1C and FIGS. 2A-2C, respectively. The results show that there are different release rates between the different ion-exchange resins.

### Example 9: Evaluation of Abuse Potential by Differential Extraction

Differential (selective) extraction of either naltrexone or methylphenidate from loaded resins using acetone, acetonitrile and pure water was examined at load amounts of about 30 mg. The methods disclosed in Examples 3-6 were used.

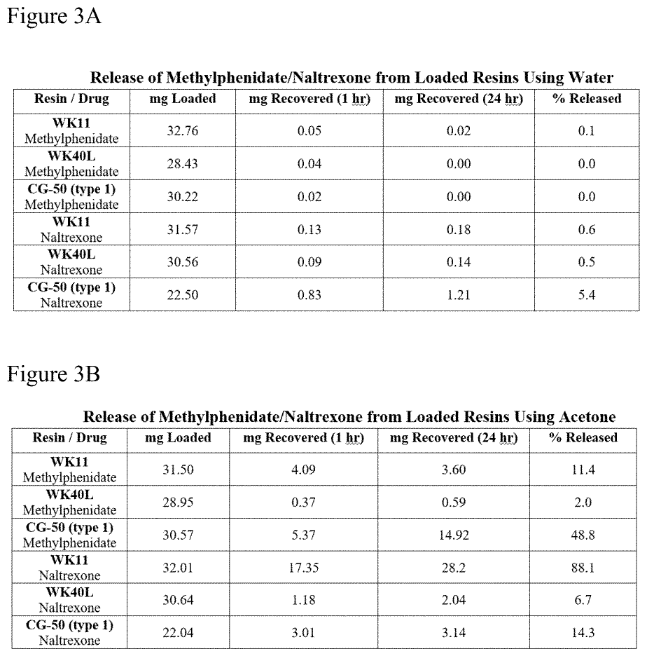

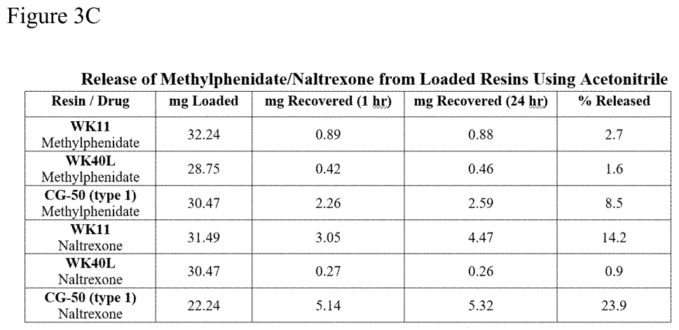

The amount of naltrexone and methylphenidate released (extracted) from the ion-exchange resins was very low using water when using a load amount of about 30 mg (see FIG. 3A). However, the results show that differential extraction from the resins using acetone or acetonitrile did take place with some resins, such as WK11 and CG-50 (see FIGS. 3B-3C).

No significant extraction of methylphenidate or naltrexone occurred from resin WK40L using water, acetone or acetonitrile, even after 24 hours (see FIGS. 3A-3C). Neither acetone, acetonitrile, nor water could overcome the drug to WK40L binding to facilitate extraction from WK40L. In contrast, both methylphenidate and naltrexone were released at high percentages from WK40L resin in SGF (see FIGS. 1A-1C and FIGS. 2A-2C). Therefore, the WK40L resin has favorable release properties and also exhibits abuse deterrence capability.

In view of the result from Example 6, lower amounts of drug loading (i.e., about 12-14 mg) showed that while resin WK100 does not retain (bind) naltrexone well in the presence of acetone, it does strongly retain methylphenidate. The results show the possibility of using WK100 to bind methylphenidate and using WK40L to bind naltrexone, in a two resin abuse-deterrent formulation.

### Example 10: Evaluation of Abuse Potential of Combination Drug Product

In order to further evaluate abuse potential in a combination drug product, extraction of each drug was examined in the presence of the other. Naltrexone and methylphenidate were separately loaded onto resins. The loaded resins were then combined in the same vial at therapeutic dosages (about 30 mg each). Naltrexone and methylphenidate were extracted together (each in the presence of the other drug) using acetone, acetonitrile or water. Samples were analyzed after 1 hour and after 24 hours. No significant amount of either drug (<1%) was extracted from the resins using acetone, acetonitrile or water after 24 hours. The detailed procedure for this example are shown below:

### Resin Loading—Methylphenidate

1. Prepare at least 75 mL of a 4.0 mg/mL stock reference solution of Methylphenidate in pH 5.0 Sodium Phosphate buffer.
2. Prepare test samples by labeling separate 50-mL centrifuge filter tubes with each resin type, compound, pH buffer, and release condition.
3. Place parafilm around the bottom of the insert and return filter to centrifuge tube.
4. Weigh 250±2.5 mg portions of dried, activated resins for each test sample and transfer to centrifuge tube.
5. Transfer 10-mL of buffer into corresponding sample tube and cap the tube.
6. Mix by stirring on rotational mixer at ambient temp. for 4 hours.
7. Take insert out of tube, remove parafilm, and return to tube.
8. Separate resins from loading solution by centrifugation and store loading solution.

### Resin Loading—Naltrexone

1. Prepare at least 75 mL of a 4.0 mg/mL stock reference solution of Naltrexone in pH 7.0 Sodium Phosphate buffer.
2. Prepare test samples by labeling separate 50-mL centrifuge filter tubes with each resin type, compound, pH buffer, and release condition. Prepare each sample in duplicate.
3. Place parafilm around the bottom of the insert and return filter to centrifuge tube.
4. Accurately weigh 250±2.5 mg portions of dried, activated resins for each test sample and transfer to centrifuge tube.
5. Transfer 10-mL of buffer into corresponding sample tube and cap the tube.
6. Mix by stirring on rotational mixer at ambient temp. for 4 hours.
7. Take insert out of tube, remove parafilm, and return to tube.
8. Separate resins from loading solution by centrifugation and store loading solution.
   Methylphenidate Naltrexone Release in Solvents with Abuse Potential 1 Hour and 24 Hours
1. Measure 500 mL of acetone into graduated cylinder.
2. Select both “A” samples (MPH and NTX) designated for acetone release.
3. Remove inserts from centrifuge tubes and transfer resins to media bottle, rinsing tube with solvent until all resins are collected in bottle.
4. Add remaining solvent to bottle and cap.
5. Repeat Steps 1-4 with each remaining set of samples.
6. Place bottles onto orbital shaker and rotate at 100 rpm at room temperature.
7. After one hour, remove a small amount of the solution (<1.0 mL) and place into HPLC vial. Filter vial may be used if necessary to remove solution from resin.
8. Allow resins to mix in solvents for an additional 23 hours and remove additional sample from vessel for HPLC analysis.
9. Determine Methylphenidate/Naltrexone sample concentration (mg/mL) by HPLC (using 0.08 mg/mL Methylphenidate/Naltrexone reference solution).
10. Calculate: mg/mL Methylphenidate/Naltrexone=peak area sample/peak area reference solution×mg/mL reference solution
11. Calculate: mg Released=mg/mL Methylphenidate/Naltrexone in sample×500 mL (solvent)
12. Calculate: % Released=mg Released (solvent)/mg Loaded (in resin)×100
   The results are presented in Table 20 below.

TABLE 20
Release of Methylphenidate/Naltrexone from Loaded Resins

| | | | | |
|---|---|---|---|---|
| | | H2O | H2O | |
| | H2O | mg | mg | H2O |
| | mg | Released | Released | % |
| Resin/Analyte | Loaded | 1 hour | 24 hours | Released |

| | | | | | |
|---|---|---|---|---|---|
| WK40L | A | 29.24 | 0.04 | 0.00 | 0.00 |
| MPH | B | 29.69 | 0.04 | 0.00 | 0.00 |
| WK40L | A | 30.24 | 0.04 | 0.12 | 0.4 |
| NTX | B | 30.34 | 0.04 | 0.12 | 0.4 |

| | | | Acetone | Acetone | | | ACN | ACN | |
|---|---|---|---|---|---|---|---|---|---|
| | | Acetone | mg | mg | Acetone | ACN | mg | mg | ACN |
| | | mg | Released | Released | % | mg | Released | Released | % |
| Resin/Analyte | | loaded | 1 hour | 24 hours | Released | Loaded | 1 hour | 24 hours | Released |
| WK40L | A | 29.52 | 0.07 | 0.33 | 1.12 | 29.60 | 0.03 | 0.04 | 0.12 |
| MPH | B | 29.52 | 0.09 | 0.29 | 0.97 | 29.33 | 0.00 | 0.02 | 0.05 |
| WK40L | A | 30.24 | 0.12 | 0.23 | 0.76 | 30.32 | 0.03 | 0.02 | 0.08 |
| NTX | B | 30.31 | 0.12 | 0.24 | 0.80 | 29.87 | 0.03 | 0.02 | 0.06 |

### Example 11: Determination of Loading Capacity and Release Rates for Pharmaceutical or Food Grade Resins

In order to identify resins that are fit for human consumption (pharmaceutical or food grade) and have suitable loading capacity and release rates, two resins (WK10S and CM400/SS) were evaluated. The resins were first screened using the orbital shaker. Then the dissolution rates were determined.

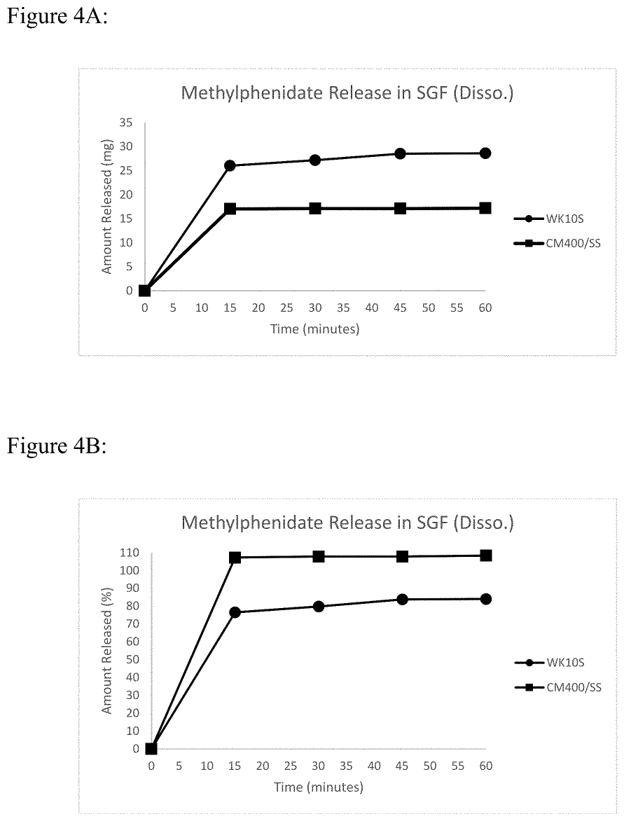

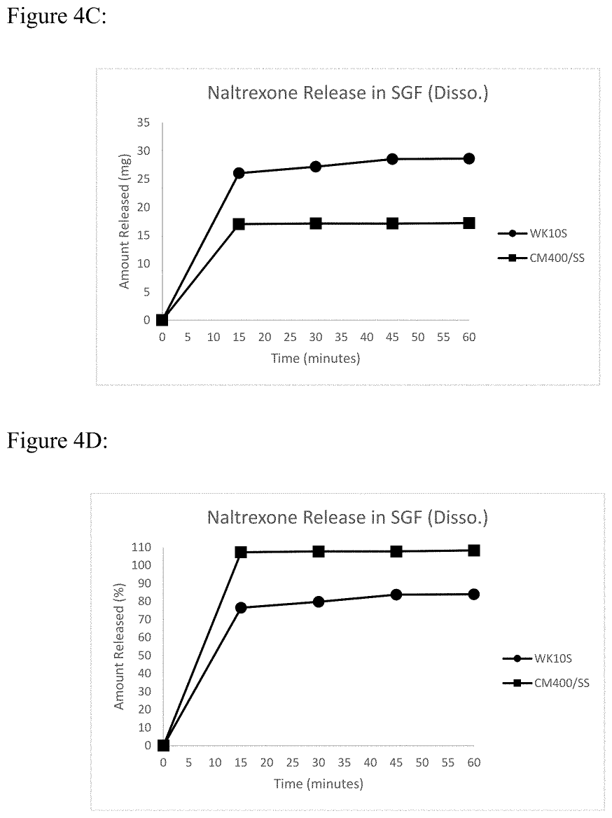

Results indicate that loading capacity and release rates for Methylphenidate and Naltrexone were suitable using the WK10S resin, loading more than 30 mg of each (using ˜250 mg resin), and releasing more than 80% in SGF within 45 minutes. The loading capacity for resin CM400/SS was not as favorable. The results for loading and release of Methylphenidate and Naltrexone are presented in Tables 21, 22, 23 and FIGS. 4A-D that show release of Methylphenidate and Naltrexone from WK10S and CM400/SS resins.

TABLE 21
Release of Methylphenidate from Loaded
Resins Using SGF After 1 Hour

| | mg | mg | % |
|---|---|---|---|
| | Methylphe- | Methylphe- | Methylphe- |
| | nidate | nidate | nidate |
| Resin/pH | Loaded | Recovered | Released |
| WK10S | 34.01 | 31.44 | 92.4 |
| Orbital Shaker A | | | |
| WK10S | 34.01 | 31.02 | 91.2 |
| Orbital Shaker B | | | |
| WK10S | 34.09 | 28.39 | 83.3 |
| Disso. App. A | | | |
| WK10S | 34.08 | 28.87 | 84.7 |
| Disso. App. B | | | |
| CM400/SS | 15.68 | 16.09 | 102.6 |
| Orbital Shaker A | | | |
| CM400/SS | 15.84 | 16.28 | 102.8 |
| Orbital Shaker B | | | |
| CM400/SS | 15.83 | 17.33 | 109.5 |
| Disso. App. A | | | |
| CM400/SS | 15.95 | 17.06 | 107.0 |
| Disso. App. B | | | |

TABLE 22
Release of Naltrexone from Loaded
Resins Using SGF After 1 Hour

| | mg | mg | % |
|---|---|---|---|
| | Naltrexone | Naltrexone | Naltrexone |
| Resin/pH | Loaded | Recovered | Released |
| WK10S | 30.64 | 29.66 | 96.8 |
| Orbital Shaker A | | | |
| WK10S | 31.11 | 29.72 | 95.5 |
| Orbital Shaker B | | | |
| WK10S | 30.88 | 26.11 | 84.5 |
| Disso. App. A | | | |
| WK10S | 30.88 | 26.98 | 87.4 |
| Disso. App. B | | | |
| CM400/SS | 18.34 | 19.74 | 107.6 |
| Orbital Shaker A | | | |
| CM400/SS | 16.70 | 17.88 | 107.1 |
| Orbital Shaker B | | | |
| CM400/SS | 18.10 | 19.45 | 107.4 |
| Disso. App. A | | | |
| CM400/SS | 18.24 | 19.77 | 108.4 |
| Disso. App. B | | | |

TABLE 23
Release of MPH and NTX using Dissolution Apparatus and SGF

| | mg | % | | |
|---|---|---|---|---|
| | Methylphe- | Methylphe- | mg | % |
| | nidate | nidate | Naltrexone | Naltrexone |
| Resin/pH | Recovered | Released | Recovered | Released |
| WK10S A | 26.34 | 77.3 | 22.73 | 73.6 |
| 15 min | | | | |
| WK10S B | 25.75 | 75.6 | 22.19 | 71.9 |
| 15 min | | | | |
| WK10S A | 27.33 | 80.2 | 24.37 | 78.9 |
| 30 min | | | | |
| WK10S B | 27.05 | 79.4 | 24.29 | 78.6 |
| 30 min | | | | |
| WK10S A | 29.08 | 85.3 | 27.09 | 87.7 |
| 45 min | | | | |
| WK10S B | 28.01 | 82.2 | 25.78 | 83.5 |
| 45 min | | | | |
| WK10S A | 28.39 | 83.3 | 26.11 | 84.5 |
| 60 min | | | | |
| WK10S B | 28.87 | 84.7 | 26.98 | 87.4 |
| 60 min | | | | |
| CM400/SS A | 17.26 | 109.1 | 19.38 | 107.0 |
| 15 min | | | | |
| CM400/SS B | 16.82 | 105.5 | 19.44 | 106.6 |
| 15 min | | | | |
| CM400/SS A | 17.30 | 109.3 | 19.45 | 107.4 |
| 30 min | | | | |
| CM400/SS B | 16.96 | 106.3 | 19.62 | 107.5 |
| 30 min | | | | |
| CM400/SS A | 17.33 | 109.5 | 19.51 | 107.8 |
| 45 min | | | | |
| CM400/SS B | 16.90 | 106.0 | 19.59 | 107.4 |
| 45 min | | | | |
| CM400/SS A | 17.33 | 109.5 | 19.45 | 107.4 |
| 60 min | | | | |
| CM400/SS B | 17.06 | 107.0 | 19.77 | 108.4 |
| 60 min | | | | |

### Example 12: Evaluation of Abuse Potential by Differential Extraction for Pharmaceutical or Food Grade Resins

Pharmaceutical/Food grade resins (WK10S, CM400/SS, and IRP-64) were also screened using abuse potential solvents. An orbital shaker set at room temperature was used during differential extraction procedures, with release of drug analyzed at 1 hour and 24 hours of mixing in target solvent. Drugs were loaded and released separately of each other. Results (Tables 24, 25, and 26) show that differential extraction does take place with the tested resins, with some solvents having a greater effect than others. WK10S and 4RP-64 tended to retain drug compound on the resin more successfully than CM400/SS. With the exception of WK10S released in acetone, all resins retained more MPH than NTX when submerged and mixed in abuse potential solvents.

TABLE 24
Release of Methylphenidate/Naltrexone from Loaded Resins in Water and Acetone

| | | H2O | H2O | | | Acetone | Acetone | |
|---|---|---|---|---|---|---|---|---|
| | H2O | mg | mg | H2O | Acetone | mg | mg | Acetone |
| | mg | Released | Released | % | mg | Released | Released | % |
| Resin/Analyte | Loaded | 1 hour | 24 hours | Released | loaded | 1 hour | 24 hours | Released |
| WT01S | 33.89 | 0.05 | 0.06 | 0.2 | 33.28 | 8.27 | 11.30 | 34.0 |
| MPH | | | | | | | | |
| CM400/SS | 16.69 | 0.62 | 0.88 | 5.3 | 16.61 | 6.08 | 7.32 | 44.1 |
| MPH | | | | | | | | |
| WT01S | 29.74 | 0.24 | 0.42 | 1.4 | 29.01 | 4.19 | 6.95 | 23.9 |
| NTX | | | | | | | | |
| CM400/SS | 16.77 | 6.32 | 7.08 | 42.2 | 16.95 | 17.69 | 19.40 | 114.5 |
| NTX | | | | | | | | |

TABLE 25
Release of Methylphenidate/Naltrexone from Loaded Resins in Acetonitrile and Isopropanol

| | | ACN | ACN | | | IPA | IPA | |
|---|---|---|---|---|---|---|---|---|
| | ACN | mg | mg | ACN | IPA | mg | mg | IPA |
| | mg | Released | Released | % | mg | Released | Released | % |
| Resin/Analyte | Loaded | 1 hour | 24 hours | Released | loaded | 1 hour | 24 hours | Released |
| WT01S | 33.24 | 1.78 | 2.47 | 7.4 | 33.21 | 0.56 | 8.51 | 25.6 |
| MPH | | | | | | | | |
| CM400/SS | 16.64 | 5.53 | 6.44 | 38.7 | 16.76 | 2.43 | 6.94 | 41.4 |
| MPH | | | | | | | | |
| WT01S | 29.69 | 3.55 | 3.81 | 12.8 | 28.54 | 0.61 | 4.26 | 14.9 |
| NTX | | | | | | | | |
| CM400/SS | 16.80 | 15.51 | 16.87 | 100.4 | 16.79 | 4.29 | 14.94 | 89.0 |
| NTX | | | | | | | | |

TABLE 26
Release of Methylphenidate/Naltrexone from Loaded Resins

| | | | H2O | H2O | H2O | | Acetone |
|---|---|---|---|---|---|---|---|
| | | H2O | mg | mg | % | Acetone | mg |
| | | mg | Released | Released | Released | mg | Released |
| Resin/Analyte | | Loaded | 1 hour | 24 hours | 24 hours | loaded | 1 hour |
| IRP-64 | A | 30.97 | 0.07 | 0.07 | 0.2 | 24.26 | 1.24 |
| MPH | B | 30.94 | 0.14 | 0.19 | 0.6 | 23.85 | 1.19 |
| IRP-64 | A | 23.98 | 0.38 | 1.67 | 7.0 | 23.37 | 1.42 |
| NTX | B | 24.22 | 1.67 | 1.67 | 6.9 | 24.14 | 5.16 |

| | | Acetone | Acetone | | ACN | ACN | ACN |
|---|---|---|---|---|---|---|---|
| | | mg | % | ACN | mg | mg | % |
| | | Released | Released | mg | Released | Released | Released |
| Resin/Analyte | | 24 hours | 24 hours | Loaded | 1 hour | 24 hours | 24 hours |
| IRP-64 | A | 6.87 | 28.3 | 27.37 | 2.86 | 5.92 | 21.6 |
| MPH | B | 9.26 | 38.8 | 29.32 | 2.23 | 6.12 | 20.9 |
| IRP-64 | A | 3.26 | 14.0 | 23.11 | 2.71 | 8.82 | 38.2 |
| NTX | B | 5.72 | 23.7 | 24.20 | 2.47 | 9.33 | 38.6 |

1. A pharmaceutical formulation comprising:
a CNS stimulant, or a pharmaceutically acceptable salt thereof,
an opioid receptor antagonist, or a pharmaceutically acceptable salt thereof, and one or more pharmaceutically acceptable resins,
wherein the CNS stimulant and the opioid receptor antagonist have substantially different solubilities in the presence of at least one solvent; and
wherein the one or more resins bind the CNS stimulant and the opioid receptor antagonist in the presence of the at least one solvent.

2. The pharmaceutical formulation of claim 1, wherein the CNS stimulant and the opioid receptor antagonist have an immediate release profile in the presence of simulated gastric fluid.

3. The pharmaceutical formulation of claim 1, wherein the CNS stimulant and the opioid receptor antagonist have an intermediate or extended release profile in the presence of simulated gastric fluid.

4. The pharmaceutical formulation of claim 1, wherein the CNS stimulant is selected from one or more of methylphenidate (MPH), amphetamine, benzphetamine, dextroamphetamine, dexmethylphenidate, diethylpropion, lisdexamfetamine, methamphetamine, armodafinil, modafinil, phendimetrazine, and phentermine, or a pharmaceutically acceptable salt thereof.

5. The pharmaceutical formulation of claim 1, wherein the opioid receptor antagonist is selected from one or more of naltrexone (NTX), nor-binaltorphimine (norBNI), nalmefene, nalodeine, samidorphan, naloxone, and 613-Naltrexol.

6. The pharmaceutical formulation of claim 1, wherein the resin comprises a weak acid cation ion exchange resin; a strong acid cation ion exchange resin; a weak basic anion ion exchange resin; a strong basic anion ion exchange resin; or a medium basic anion exchange resin.

7. The pharmaceutical formulation of claim 6, wherein the resin comprises a polymer or copolymer of one or more of acrylate, methacrylate, divinylbenzene (DVB), and polystyrene.

8. The pharmaceutical composition of claim 7, wherein the resin comprises a polymer backbone selected from acrylic, methacrylic, methacrylic-DVB copolymer, styrene-DVB co-polymer; and acryl-DVB copolymer.

9. The pharmaceutical composition of claim 8, wherein the polymer is crosslinked.

10. The pharmaceutical formulation of claim 6, wherein the resin comprises one or more functional groups selected from: primary amine, secondary amine, tertiary amine, quaternary amine, carboxylic acid, sulfonic acid, thiol, imiodiacetic acid, aminophosphonic acid, thiourea, bis-picolylamine, dithiocarbamate, thiouronium, amidoxime, and N-methyl glucamine.

11. The pharmaceutical formulation of claim 6, wherein the resin has a size in the range of about 50 and 500 mesh.

12. The pharmaceutical formulation of claim 1, wherein the resin is in the form of a powder, a gel, or beads.

13. The pharmaceutical formulation of claim 1, wherein the resin is macroporous or microporous.

14. The pharmaceutical formulation of claim 13, wherein the resin is isoporous.

15. The pharmaceutical formulation of claim 1, wherein the solvent in which the CNS stimulant and the opioid receptor antagonist have substantially different solubilities is selected from: acetone, acetonitrile, isopropyl alcohol, methyl ethyl ketone, hexane, water, and ethyl acetate.

16. The pharmaceutical formulation of claim 1, wherein the stimulant is methylphenidate or amphetamine, or pharmaceutically acceptable salts thereof, and the opioid receptor antagonist is naltrexone, or a pharmaceutically acceptable salt thereof.

17. The pharmaceutical formulation of claim 16, wherein the resin is a weakly acidic cation exchange resin, and which has a polyacrylate backbone and a carboxylic acid or carboxylate group as ionizable groups.

18. A method for making an abuse deterrent pharmaceutical formulation, comprising:
loading a CNS stimulant and an opioid receptor antagonist on one or more pharmaceutically acceptable resins,
wherein the CNS stimulant and the opioid receptor antagonist have substantially different solubilities in the presence of at least one solvent; and
wherein the one or more resins bind the CNS stimulant and the opioid receptor antagonist in the presence of the at least one solvent.

19-34. (canceled)
